(19) United States
(12) Patent Application Publication
(10) Pub. No.: US 2015/0107993 A1
(43) Pub. Date: Apr. 23, 2015

(54) METHODS AND APPARATUSES FOR EVALUATING WATER POLLUTION

(71) Applicant: Transfert Plus, s.e.c., Montreal (CA)

(72) Inventors: Ricardo Izquierdo, Montreal-Ouest (CA); Philippe Juneau, Longueuil (CA); Florent Lefèvre, Montreal (CA)

(21) Appl. No.: 14/396,218

(22) PCT Filed: Apr. 18, 2013
(86) PCT No.: PCT/CA2013/000383

§ 371 Date: Oct. 22, 2014

Related U.S. Application Data
(60) Provisional application No. 61/637,546, filed on Apr. 24, 2012.

(51) Int. Cl. G01N 33/18; G01N 27/30; G01N 21/64

(52) U.S. Cl. CPC G01N 33/1866; G01N 21/64; G01N 27/30; G01N 2201/068; G01N 2201/0628
USPC 204/403.01

(57) ABSTRACT

There are provided methods and apparatuses for evaluating water pollution. The apparatus comprises at least one light source for exciting or causing activity of at least one type of microorganism or biological material; at least one photodetector for detecting a level of fluorescent light; and a chip disposed between the at least one light source and the detector, the chip comprising at least one microfluidic channel disposed for being exposed to light from the at least one light source and dimensioned for receiving a composition comprising the at least one type of microorganism or biological material and a water sample to be evaluated.

### CROSS-REFERENCE TO RELATED APPLICATIONS

The present disclosure claims the benefit of priority from U.S. provisional application No. 61/637,546 filed on Apr. 24, 2012, the content of which is herein incorporated by reference in its entirety.

### FIELD OF THE DISCLOSURE

The present disclosure relates to the field of evaluating pollution in a water sample. In particular, the present disclosure relates to apparatuses and methods for evaluating pollution in a water sample using microorganisms.

### BACKGROUND OF THE DISCLOSURE

Several systems and methods are known in the art for evaluating pollution in a water sample using microorganisms. However, several of them are either very costly to acquire and/or to operate. Moreover, several of them require cumbersome equipment. Several of them further require a long time for completing an evaluation, often in the magnitude of hours or days.

### SUMMARY OF THE DISCLOSURE

It would thus be highly desirable to be provided with an apparatus or a method that would at least partially solve one of the problems previously mentioned or that would be an alternative to the existing technologies.

According to one aspect, there is provided an apparatus for evaluating an analyte comprising:
at least one light source for emitting light having a spectral range for exciting at least one biological material or microorganism or at least one organic or inorganic compound;
at least one photodetector for detecting a level of fluorescent light;
a chip disposed between the at least one light source and the detector, the chip comprising at least one microfluidic channel disposed for being exposed to light from the at least one light source and dimensioned for receiving a composition comprising the at least one type of photosynthetic microorganism and a water sample to be evaluated;
an electric detector comprising at least two electrodes positioned in the at least one microfluidic channel for detecting at least one property of the composition; and
   wherein the detected level of fluorescent light provides a first indication of concentration of at least one compound in the analyte and the at least one detected property of the composition provides a second indication of the pollution level of the water sample.

According to one aspect, there is provided an apparatus for evaluating water pollution comprising:
at least one light source for emitting light having a spectral range for causing at least one type of photosynthetic microorganism to undergo cell photoactivity (for example photosynthesis);
at least one photodetector for detecting a level of fluorescent light;
a chip disposed between the at least one light source and the detector, the chip comprising at least one microfluidic channel disposed for being exposed to light from the at least one light source and dimensioned for receiving a composition comprising the at least one type of photosynthetic microorganism and a water sample to be evaluated;
an electric detector comprising at least two electrodes positioned in the at least one microfluidic channel for detecting at least one property of the composition; and
   wherein the detected level of fluorescent light provides a first indication of pollution level in the water sample and the at least one detected property of the composition provides a second indication of the pollution level of the water sample.

According to another aspect, there is provided a chip for receiving microorganism or biological material comprising:
a substrate defining at least one microfluidic channel for receiving a composition comprising an analyte and at least one type of microorganism or biological material, the at least one microfluidic channel further defining at least one microfluidic chamber, the substrate being substantially transparent at the location of the microfluidic chamber;
a filter that is at least substantially semi-transparent and that is supported within the microfluidic chamber, the filter substantially preventing passage of the microorganism or biological material while permitting flow of the water sample therethrough, the filter being aligned with a substantially transparent portion of the substrate;

at least two electrodes positioned within the microfluidic channel for taking electrical measurements.

According to another aspect, there is provided a chip for receiving microorganism or biological material comprising:
a substrate defining at least one microfluidic channel for receiving a composition comprising a water sample and at least one type of microorganism or biological material, the at least one microfluidic channel further defining at least one microfluidic chamber, the substrate being substantially transparent at the location of the microfluidic chamber;
a filter that is at least substantially semi-transparent and that is supported within the microfluidic chamber, the filter substantially preventing passage of the microorganism or biological material while permitting flow of the water sample therethrough, the filter being aligned with a substantially transparent portion of the substrate;
at least two electrodes positioned within the microfluidic channel for taking electrical measurements.

According to another aspect, there is provided an apparatus for evaluating at least one analyte comprising:
at least one light source for emitting light;
at least one photodetector for detecting a light; and
a chip defining a chip plane disposed between to the at least one light source and the at least one detector, the chip comprising at least one microfluidic channel for receiving a composition comprising the at least one the analyte and at least one type of microorganism or biological material, the at least one microfluidic channel defining a microfluidic chamber being exposed to light from the at least one light source; and
an electric detector comprising at least two electrodes, at least one of the electrodes being positioned within the at least one microfluidic chamber for detecting at least one property of the composition in the microfluidic chamber;
   wherein the at least one photodetector and the at least one microfluidic chamber are substantially aligned together, the light source being disposed for emitting light onto the microfluidic chamber and light emitted from the microfluidic chamber being detected by the photodetector, and wherein the at least two electrodes being effective for detecting at least one property of the composition in the aligned microfluidic chamber.

According to another aspect, there is provided an apparatus for evaluating water pollution comprising:
at least one light source for emitting light;
at least one photodetector for detecting a light; and
a chip defining a chip plane disposed between to the at least one light source and the at least one detector, the chip comprising at least one microfluidic channel for receiving a composition comprising a water sample and at least one type of microorganism or biological material, the at least one microfluidic channel defining a microfluidic chamber being exposed to light from the at least one light source; and
an electric detector comprising at least two electrodes, at least one of the electrodes being positioned within the at least one microfluidic chamber for detecting at least one property of the composition in the microfluidic chamber;
wherein the at least one photodetector and the at least one microfluidic chamber are substantially aligned together, the light source being disposed for emitting light onto the microfluidic chamber and light emitted from the microfluidic chamber being detected by the photodetector, and wherein the at least two electrodes being effective for detecting at least one property of the composition in the aligned microfluidic chamber.

According to another aspect, there is provided an apparatus for evaluating an analyte comprising:
a chip defining a thickness of less than about 20 mm the chip comprising at least one microfluidic channel for receiving a composition comprising the analyte and at least one type of microorganism or biological material;

an electric detector comprising at least two electrodes positioned in the microfluidic channel for detecting at least one property of the composition in the microfluidic channel, the at least one detected property providing an indication of concentration of at least one compound present in the analyte.

According to another aspect, there is provided an apparatus for evaluating water pollution comprising:
a chip defining a thickness of less than about 20 mm the chip comprising at least one microfluidic channel for receiving a composition comprising a water sample and at least one type of microorganism or biological material;
an electric detector comprising at least two electrodes positioned in the microfluidic channel and connected to an electric detector for detecting at least one property of the composition in the microfluidic channel, the at least one detected property providing an indication of pollution level of the water sample.

According to another aspect, there is provided an apparatus for evaluating an analyte comprising:
a chip defining a thickness of less than about 20 or 15 mm the chip comprising at least one microfluidic channel for receiving a composition comprising the analyte and at least one type of microorganism or biological material;

an electric detector comprising at least two electrodes positioned in the microfluidic channel for detecting at least one property of the composition in the microfluidic channel, the at least one detected property providing an indication of concentration of at least one compound present in the analyte.

According to another aspect, there is provided an apparatus for evaluating water pollution comprising:
a chip defining a thickness of less than about 20 or 15 mm, the chip comprising at least one microfluidic channel for receiving a composition comprising a water sample and at least one type of microorganism or biological material;
an electric detector comprising at least two electrodes positioned in the microfluidic channel and connected to an electric detector for detecting at least one property of the composition in the microfluidic channel, the at least one detected property providing an indication of pollution level of the water sample.

According to another aspect, there is provided an apparatus for evaluating an analyte comprising:
at least one light source for exciting at least one biological material, biological organism, organic compound or inorganic compound;
at least one photodetector for detecting a level of fluorescent light; and
a chip disposed between the at least one light source and the at least one photodetector, the chip comprising at least one microfluidic channel being exposed to light from the at least one light source and for receiving the and the at least one at least one biological material, biological organism, organic compound or inorganic compound;
   wherein the detected level of fluorescent light provides an indication of indication of concentration of at least one compound present in the analyte.

According to another aspect, there is provided an apparatus for evaluating water pollution comprising:
at least one light source for emitting light having a spectral range for at least one type of photosynthetic microorganism to undergo photosynthesis and emit excess energy as fluorescent light;
at least one photodetector for detecting a level of fluorescent light; and
a chip disposed between the at least one light source and the at least one photodetector, the chip comprising at least one microfluidic channel being exposed to light from the at least one light source and for receiving a water sample and the at least one type of photosynthetic microorganisms;
wherein the detected level of fluorescent light provides an indication of pollution level in the received water sample.

According to another aspect, there is provided a method for evaluating an analyte, the method comprising:
mixing a known quantity of at least a type of microorganism or biological material with the analyte in a microfluidic chamber of a chip to form a composition;
filtering the composition through a filter disposed in the microfluidic chamber to collect the at least one microorganism or biological material at the filter;
exposing the composition in the microfluidic chamber to a light source;
detecting a level of light emitted from the microfluidic chamber; and
detecting with an electric detector at least one electrical property of the composition within the microfluidic chamber;

wherein the detected level of light provides a first indicator of level of concentration of at least one compound in the analyte and the detected at least one electrical property of the composition provides at least one further indicator of level of concentration of the at least one compound in the analyte.

According to another aspect, there is provided a method for evaluating pollution a water sample, the method comprising:
mixing a known quantity of at least a type of microorganism or biological material with the water sample in a microfluidic chamber of a chip to form a composition;
filtering the composition through a filter disposed in the microfluidic chamber to collect the at least one microorganism or biological material at the filter;
exposing the composition in the microfluidic chamber to a light source;
detecting a level of light emitted from the microfluidic chamber; and
detecting with an electric detector at least one electrical property of the composition within the microfluidic chamber;
wherein the detected level of light provides a first indicator of level of pollution of the water sample and the detected at least one electrical property of the composition provides at least one further indicator of level of pollution.

According to another aspect, there is provided a method for evaluating an analyte, the method comprising:
mixing together at least one type of photosynthetic microorganism having a known concentration and the analyte to form a composition;
emitting a light onto the composition, the light having a spectral range for causing the at least one type of photosynthetic microorganism to undergo photosynthesis and emit excess energy as fluorescent light;
detecting a level of the fluorescent light emitted by the at least one type of photosynthetic microorganism, the detected level of fluorescent light providing an indication of concentration of at least one compound present in the analyte.

According to another aspect, there is provided a method for evaluating pollution in a water sample, the method comprising:
mixing together at least one type of photosynthetic microorganism having a known concentration and the water sample to form a composition;
emitting a light onto the composition, the light having a spectral range for causing the at least one type of photosynthetic microorganism to undergo photosynthesis and emit excess energy as fluorescent light;
detecting a level of the fluorescent light emitted by the at least one type of photosynthetic microorganism, the detected level of fluorescent light providing an indication of pollution level in the water sample.

According to another aspect, there is provided a slide for holding at least one type of microorganism or biological material comprising:
a first substrate having at least one substantially transparent portion;
a second substrate having at least one substantially transparent portion aligned with the transparent portion of the first substrate;
a permeable layer disposed between the first substrate and the second substrate, the permeable layer defining at least one microfluidic chamber being aligned with the at least one transparent portion of each of the first and second substrates, the microfluidic chamber entrapping at least one type of microorganism or biological material.

According to another example, there is provided an apparatus for evaluating an analyte comprising:
at least one light source connected to a housing of the apparatus; and
at least one photodetector, connected to the housing, and substantially aligned with the at least one light source, the at least one photodetector and the at least one light source defining a space therebetween that is adapted to receive a slide containing a composition to be evaluated and comprising the analyte at least one type of microorganism or biological material.

According to another example, there is provided an apparatus for evaluating water pollution comprising:
at least one light source connected to a housing of the apparatus; and
at least one photodetector, connected to the housing, and substantially aligned with the at least one light source, the at least one photodetector and the at least one light source defining a space therebetween that is adapted to receive a slide containing a composition to be evaluated and comprising a water sample at least one type of microorganism or biological material.

According to another aspect, there is provided a slide for receiving microorganism or biological material comprising:
a rigid substrate defining at least one microfluidic recess having at least one type of microorganism or biological material being held therein, the substrate being substantially transparent at least at one location defining the microfluidic recess;
a filter covering the at least one microfluidic recess for holding the at least one type of microorganism or biological material held the microfluidic recess;
at least one electrode effective for taking at least one electrical measurement, the at least one electrode being connected to the microfluidic recess and/or to the filter, the electrode comprising a nanomaterial, the nanomaterial being arranged in a plurality of members defining a plurality of pores for allowing passage of light therethrough.

According to another aspect, there is provided a kit for evaluating an analyte comprising:
a slide defining at least one microfluidic chamber for receiving a composition comprising the analyte and at least one microorganism or biological material; and
an apparatus comprising;
   at least one light source connected to a housing of the apparatus; and
   at least one photodetector connected to the housing, and substantially aligned with the at least one light source, the at least one photodetector and the at least one light source defining a space therebetween that is adapted to receive a slide containing a composition to be evaluated and comprising the analyte and at least one type of microorganism or biological material.

According to another aspect, there is provided a kit for evaluating water pollution comprising:
a slide defining at least one microfluidic chamber for receiving a composition comprising a water sample and at least one microorganism or biological material; and
an apparatus comprising;
   at least one light source connected to a housing of the apparatus; and
   at least one photodetector connected to the housing, and substantially aligned with the at least one light source, the at least one photodetector and the at least one light source defining a space therebetween that is adapted to receive a slide containing a composition to be evaluated and comprising a water sample at least one type of microorganism or biological material.

According to another aspect, there is provided a method of evaluating an analyte comprising:
inserting at least one type of microorganism or biological material and the analyte into a microfluidic chamber of a slide that is substantially transparent at the location of the microfluidic chamber;
inserting the slide between at least one light source and at least one photodetector;
substantially aligning the microfluidic chamber of the slide with the at least one light source and at least one photodetector;
emitting light from the at least one light source onto the microfluidic chamber;
detecting light emitted from the microfluidic chamber with at least one photodetector;
measuring at least one electrical property of a composition comprising the analyte and the at least one microorganism or biological material using at least one semi-transparent electrode located proximate the microfluidic chamber.

According to another aspect, there is provided a method of evaluating pollution in a water sample comprising:
inserting at least one type of microorganism or biological material and a water sample into a microfluidic chamber of a slide that is substantially transparent at the location of the microfluidic chamber;
inserting the slide between at least one light source and at least one photodetector;
substantially aligning the microfluidic chamber of the slide with the at least one light source and at least one photodetector;
emitting light from the at least one light source onto the microfluidic chamber;
detecting light emitted from the microfluidic chamber with at least one photodetector;
measuring at least one electrical property of a composition comprising the water sample and the at least one microorganism or biological material using at least one semi-transparent electrode located proximate the microfluidic chamber.

According to another aspect, there is provided an electronic detector comprising:
a working electrode;
a counter electrode; and
a reference electrode;
wherein at least one of the electrodes comprises a plurality of nanofilaments defining a plurality of pores.

According to another aspect, there is provided an electronic detector for detecting an oxygen concentration comprising:
a working electrode;
a counter electrode; and
a reference electrode;
wherein at least one of the electrodes comprises a plurality of nanofilaments defining a plurality of pores.

### BRIEF DESCRIPTION OF THE DRAWINGS

The following drawings represents non-limitative examples in which:

FIG. 1 is an exploded view of an example of an apparatus according to the present disclosure;

FIG. 2 is a side cross-section view of another example of an apparatus according to the present disclosure;

FIG. 3 is a side cross-section view of another example of an apparatus according to the present disclosure;

FIG. 4 is a side cross-section view of another example of an apparatus according to the present disclosure;

FIG. 5 is a side cross-section view of another example of an apparatus according to the present disclosure;

FIG. 5A is a plan view of an example of an electric detector according to the present disclosure;

FIGS. 6A, 6B, 6C, 6D are side cross-section views of another example of an apparatus according to the present disclosure, each figures showing different sates if the apparatus when in use;

FIG. 7 is a side cross-section view of another example of an apparatus according to the present disclosure;

FIG. 8 is a side cross-section view of another example of an apparatus according to the present disclosure;

FIGS. 9A and 9B are a side section views of examples of slides for evaluating a level of pollution in water according to the present disclosure;

FIG. 10 is a top view of another example of a slide for evaluating a level of pollution in water according to the present disclosure;

FIG. 11 is a side cross-section view of another example of a slide for evaluating a level of pollution in water according to the present disclosure;

FIG. 12 is a side cross-section view of another example of a slide for evaluating a level of pollution in water according to the present disclosure;

FIGS. 13 and 14 show the slide of FIG. 12 when being in use;

FIG. 15A is an algae absorption spectrum according to an example of the present disclosure;

FIG. 15B is an algae emission spectrum according to an example of the present disclosure;

FIG. 16 is a graph showing filter transparency as a function of the wavelength according to an example of the present disclosure;

FIG. 17 is a transmission spectra according to an example of the present disclosure;

FIG. 18A is graph showing the fluorescence signal as a function of time in another example of the present disclosure;

FIG. 18B is graph showing the fluorescence area as a function of algal concentration in another example of the present disclosure;

FIG. 19A is graph showing the fluorescence signal as a function of time in another example of the present disclosure;

FIG. 19B is graph showing variation of the inhibition factor as function of Diuron concentration;

FIG. 20 is a plan view of a plurality of electric detectors formed according to an example test apparatus;

FIG. 21A is a graph showing transparency levels of different resistivity over a range of wavelengths;

FIG. 21B is a graph showing sheet resistance for different transparency levels;

FIG. 21C is a graph showing transparency of an electrode over a range of wavelengths;

FIG. 21D is a photograph taken with an scanning electrode microscope of an electrode of a test apparatus;

FIG. 21E is a graph of variations of the size of pores over different number of pores;

FIG. 22A is a graph of oxygen concentration levels measured for a reference and for solution having Diuron; and

FIG. 22B is a graph showing oxygen concentration levels measured by a test apparatus and by a commercially available device.

### DETAILED DESCRIPTION OF THE DISCLOSURE

The expression “semi-transparent” as used herein when used to describe a material or an element, refers to a material or element that allows passage of at least 40%, 50% or 60% in the about 390 nm to about 800 nm wavelength range.

The expression “substantially transparent” as used herein when used to described a material or an element, refers to a material or element that allows passage of at least 80%, 90% or 95% in the about 390 nm to about 800 nm wavelength range.

The apparatuses, methods, kits and slides of the present disclosure are effective for carrying out various analyses on various types of analytes (such as various liquids comprising at least one organic or inorganic or water comprising at least one pollutant) for example by using at least one microorganism or at least biological material. The at least one microorganism can be at least one type of photosynthetic microorganism. The at least one biological material can be an organic compound, a pigment, a photo-sensible biological material. For example, the biological material can be a non-photosynthetic organism, sub-part of photosynthetic or non-photosynthetic organisms such as organelles or intact cells.

For example, microorganism can be microalgae, cyanobacteria, and photosynthetic bacteria, or biological material containing or not pigments (such as chlorophylls, carotenoids, phycoerythrin and phycocyanin).

For example, the at least one type of photosynthetic microorganism can be chosen from microalgae, cyanobacteria and photosynthetic bacteria.

For example, the at least one microfluidic channel can define at least one microfluidic chamber, the at least one chamber comprising a filter substantially preventing passage of the microorganisms or biological material while permitting flow of the water sample therethrough; and the at least one of the electrodes comprised in the electric detector is positioned within the at least one microfluidic chamber.

For example, the electrodes can detect at least one electrical property of the composition in the microfluidic chamber.

For example, the filter can be at least semi-transparent.

For example, the at least one photodetector, the at least one microfluidic chamber, and the filter can be substantially aligned together.

For example the at least one light source can be aligned with the at least one photodetector.

For example, the chip can define a chip plane, the filter can be at least semi-transparent; and the at least one photodetector, the at least one microfluidic chamber, and the filter can be substantially aligned in a direction transverse the chip plane.

For example, the filter can be substantially transparent.

For example, at least one of the electrodes can comprise a nanomaterial being connected to the filter, the nanomaterial being arranged in a plurality of members defining a plurality of pores for allowing passage of light and/or water therethrough.

For example, at least one of the electrodes can be semi-transparent.

For example, at least one of the electrodes can be porous.

For example, the at least one electrode can comprise a plurality of nanomaterial members defining a plurality of pores.

For example, the at least one electrode can be formed of a plurality of nanomaterial members defining a plurality of pores.

For example, the at least one of the electrodes can have a transparency greater than about 60%, about 65% or about 70%.

For example, the resistance of the at least one of the electrodes can be less than about 10 ohms/square or less than about about 20 ohms/square and the transparency can be less than about 65%, about 75% or about 80%.

For example, the nanomaterial members can be nanofilaments that are formed of silver.

For example, the nanofilaments can be coated with platinum, nickel copper, gold or mixtures thereof.

For example, at least one electrode can be coated with platinum, nickel, copper, gold or mixtures thereof.

For example the resistance of the at least one electrode can be of about 50% to about 70% and the transparency of the at least one electrode can be about 8 ohms/square to about 30 ohms/square.

For example, the at least one property detected by the electric detector can be chosen from current, voltage, resistivity, capacity and conductivity.

For example the at least one property detected by the electric detector can be oxygen concentration.

For example, the electric detector can comprise a working electrode, a counter electrode; and a reference electrode; and each of the electrodes can be formed of a plurality of nanofilaments defining a plurality of pores.

For example, the nanofilaments can be formed of silver; and the nanofilaments forming the working electrode and the counter electrode can be coated with platinum.

For example, at least the working electrode can be aligned with the light source.

For example, at least one microfluidic channel can define a first opening, whereby when the apparatus is submerged in a volume water, the water sample can enter through the first opening to be received in the at least one microfluidic channel.

For example, the apparatus can further comprise a first optical filter disposed between the chip and the at least one photodetector, the first optical filter having a passband corresponding to the spectral range of fluorescent light emitted by the at least one type of microorganism or biological material.

For example, the spectral range of light exposing the microfluidic channel can be different from a spectral range of the fluorescent light emitted by the at least one type of microorganism or biological material.

For example, the at least one microfluidic channel can have a depth of less than about 2 mm.

For example, the at least one microfluidic channel can have a depth of less than about 1 mm.

For example, the chip can define a thickness of less than about 10 or 5 mm.

For example, the apparatus can further comprise a substrate supporting the at least one light source, a second optical filter disposed between the substrate and the chip, the second optical filter having a passband corresponding to the spectral range for causing the at least one type of microorganism or biological material to undergo cell activity and emit fluorescent light.

For example, the at least one light source can be at least one organic light emitting diodes.

For example, the at least one type of microorganism can comprise at least one type of photosynthetic microorganism.

For example, the at least one type of biological material can contain pigments.

For example, the at least one microfluidic channel can comprise the at least one type of microorganism entrapped therein.

For example, the at least one microfluidic channel can comprise the at least one type of biological material entrapped therein.

For example, at least the working electrode can be positioned within the microfluidic chamber.

For example, the apparatus for evaluating water pollution comprising the chip can further comprise at least one light source for emitting light; and at least one photodetector for detecting a light and the apparatus can be adapted to receive the chip between the at least one light source and the at least one photodetector.

For example, the at least one type of microorganism or biological material can be at least one type of photosynthetic microorganism and the at least one light source can emit light having a spectral range for causing the at least one type of photosynthetic microorganism to undergo photosynthesis and emit excess energy as fluorescent light; and the detector can be adapted for detecting a level of fluorescent light, the detected level of fluorescent light providing an additional indication of level of pollution of the water sample.

For example, the at least one photodetector, the at least one microfluidic chamber and the at least one light source can be substantially aligned together, the at least one light source being effective for emitting light onto the microfluidic chamber and light emitted from the aligned microfluidic chamber being detected by the photodetector, and the at least two electrodes can be effective for detecting the at least one property of the composition in the aligned microfluidic chamber, thereby allowing for measuring simultaneously a first indication of pollution level in the water sample by means of the at least one photodetector and a second indication of the pollution level of the water sample by means of the at least one detected property of the composition detected by the at least one electric detector.

For example, the microfluidic chamber comprises a filter that can substantially prevent passage of the at least one type of microorganism or biological material, the filter of microfluidic chamber being at least semi-transparent so as to allow passage of the light from the at least one light source therethrough.

For example, the filter can be substantially transparent.

For example, at least one detected electrical property can indicate an oxygen concentration level.

For example, the method for evaluating pollution in a water sample can further comprise determining a level of the pollution based on the detected level of fluorescent light, the known concentration of microorganism and the type of photosynthetic microorganism.

For example, the spectral range of the light emitted onto the composition can be different from a spectral range of the fluorescent light emitted by the at least one type of photosynthetic microorganism.

For example mixing the at least one type of photosynthetic microorganism and the water sample can comprise inserting a first type of photosynthetic microorganism and the water sample into a first microfluidic channel of a chip.

For example, the method for evaluating pollution in a water sample can further comprise inserting a second type of photosynthetic microorganism and a second water sample into a second microfluidic channel of the chip, thereby having a second composition into the second microfluidic channel, emitting the light onto the second composition, the light having a spectral range for causing the second type of photosynthetic microorganism to undergo photosynthesis and emit excess energy as fluorescent light; and detecting a level of the fluorescent light emitted by the second type of photosynthetic microorganism, the detected level of fluorescent light providing an indication of pollution level in the second water sample.

For example, the type of the first photosynthetic microorganism and the type of the second photosynthetic microorganism are different.

For example, concentration of the first type of photosynthetic microorganism and concentration of the second type of photosynthetic microorganism can be different.

For example, the method of evaluating water pollution can further comprise filtering the composition through a filter of the microfluidic chamber to collect the at least one type of photosynthetic microorgansim at the filter and detecting with an electric detector at least one electrical property of the composition within the microfluidic chamber.

For example, emitting the light can comprise emitting a light having a plurality of frequencies and filtering the emitted light with at least one optical filter having a passband corresponding to the spectral range for causing the at least one type of photosynthetic microorganism to undergo photosynthesis and emit excess energy as fluorescent light.

For example, the level of fluorescent light can be detected by at least one photodetector and detecting the level of the fluorescent light can comprise prior to detecting, filtering light received at the photodetector using at least one optical filter having a passband corresponding to a wavelength range of fluorescent light emitted by the at least one type of photosynthetic microorganism; and detecting the level of the fluorescent light using the at least one photodetectors.

For example, the slide can further comprise at least one light source coupled to the first substrate for emitting light through the at least one substantially transparent portion of the first substrate into the microfluidic chamber and at least one photodetector coupled to the second substrate and aligned with the substantially transparent portion of the second substrate for detecting light being emitted from the microfluidic chamber.

For example, the light source of the slide can be aligned with the at least one substantially transparent portion of the first substrate.

For example, the slide can further comprise at least one electrode for taking at least one electrical measurement, the at least one electrode comprising a nanomaterial, the nanomaterial being arranged in a plurality of members defining a plurality of pores for allowing passage of light and water therethrough.

For example, the slide can comprise a plurality of electrodes and the slide can further comprise at least one conductive line connecting the plurality of electrodes to an input-output lead.

For example, the first and second substrates of the slide can define at least one opening, the permeable layer having at least one region being in fluid flow communication with the at least one opening, and liquid contacting the exposed region can permeate through the permeable layer to be received within the microfluidic chamber.

For example, at least one of the first and second substrates can define at least one opening, the permeable layer can have at least one region being in fluid flow communication with the at least one opening, liquid contacting an exposed region can permeate through the permeable layer to be received within the microfluidic chamber.

For example, the liquid can permeate through the permeable layer by capillary movement.

For example, the at least one of the first and second substrates that defines the at least one opening can be at least partially covered by a first membrane effective for preventing solid particles of a predetermined size from entering into the at least one opening.

For example, the first membrane can be covered by a second membrane, the second membrane being permeable to gases but being impermeable to liquids.

For example, an apparatus for evaluating water pollution can further comprise an input-output port being connected to the at least one light source and the at least one photodetector, the input-output port receiving control signals for controlling the light source and for outputting information on light detected by the photodetector.

For example, an apparatus for evaluating water pollution can further comprise at least one input-output lead for contacting a corresponding input-output lead of the slide being received in the space.

For example, the can further comprise at least one electrode for taking at least one electrical measurement.

For example, the apparatus can further comprise at least one electrode for taking at least one electrical measurement, the at least one electrode comprising a nanomaterial, the nanomaterial being arranged in a plurality of members defining a plurality of pores for allowing passage of light therethrough.

For example, the slide for receiving at least one type of microorganism or biological material can further comprise a first detachable membrane coupled to the rigid substrate and covering the at least one microfluidic recess, the first detachable membrane having at least one porous portion for permitting flow of liquid therethrough and substantially preventing flow of particles larger than the at least one type of microorganism or biological material therethrough.

For example, the slide can further comprise a second detachable membrane coupled to the first detachable membrane, the second detachable permitting passage of air into the microfluidic recess and substantially preventing flow of liquid for entering into the microfluidic recess.

For example, the kit can further comprise an input-output port being connected to the at least one light source and the at least one photodetector, the input-output port receiving control signals for controlling the light source and for outputting information on light detected by the photodetector.

For example, the kit can further comprise at least one input-output lead for contacting a corresponding input-output lead of the slide being received in the space.

For example, the kit can further comprise at least one electrode for taking at least one electrical measurement.

For example, the kit can further comprise at least one electrode for taking at least one electrical measurement, the at least one electrode comprising a nanomaterial, the nanomaterial being arranged in a plurality of members defining a plurality of pores for allowing passage of light therethrough.

For example, the at least one property detected can be chosen from concentration of O₂, H₂O₂, OH⁻, H⁺, enzyme(s), free radicals, H₂, or CO₂. It can also be concentration of pollutants or conductivity variation.

The following examples are presented in a non-limitative manner.

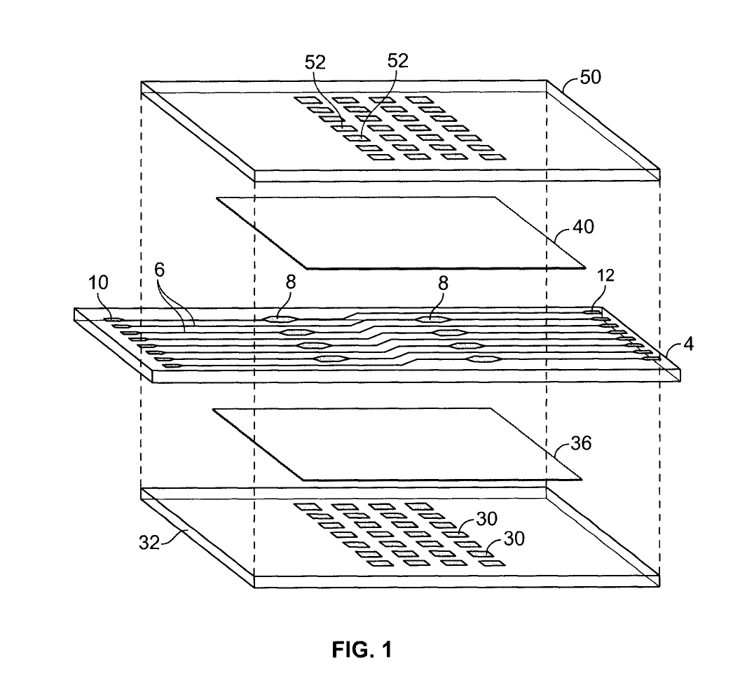

Referring now to FIG. 1, therein illustrated is an exploded view of the apparatus 2. For example, the apparatus 2 can comprise a chip 4.

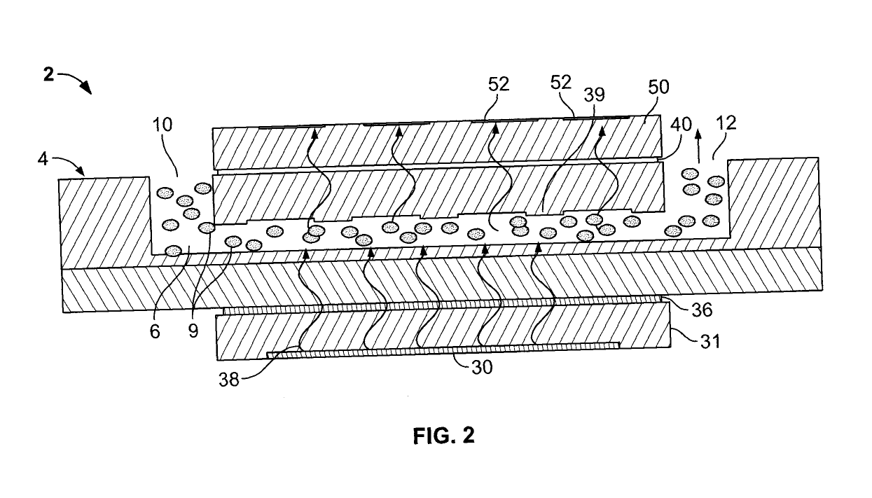

Referring now to FIG. 2, therein illustrated is a side section view of exemplary embodiments of the apparatus 2. For example, the chip 4 can comprise at least one microfluidic channels 6. For example, the microfluidic channels 6 are hollow and can extend a portion of the length of the chip 4. For example, the chip 4 can be a microelectromechanical systems (MEMS) formed of polydimenthylsiloxane material. The chip 4 can also be formed of epoxy resin, such as SU8-Microchem type, glass, or other suitable materials that allows forming of channels 6. The microfluidic channels can be fabricated using standard soft lithography techniques. However other known techniques for forming suitable microfluidic channels 6 are hereby contemplated, and such techniques are intended to be covered by the present description. FIG. 2 shows the cross section of the length of one microfluidic channel 6.

For example, the microfluidic channels 6 can be fabricated to have a depth in the micrometer range, up to 1 mm. For example, the chip 4 can be fabricated on a gas slide having a thickness in the millimeter range, which provides mechanical strength.

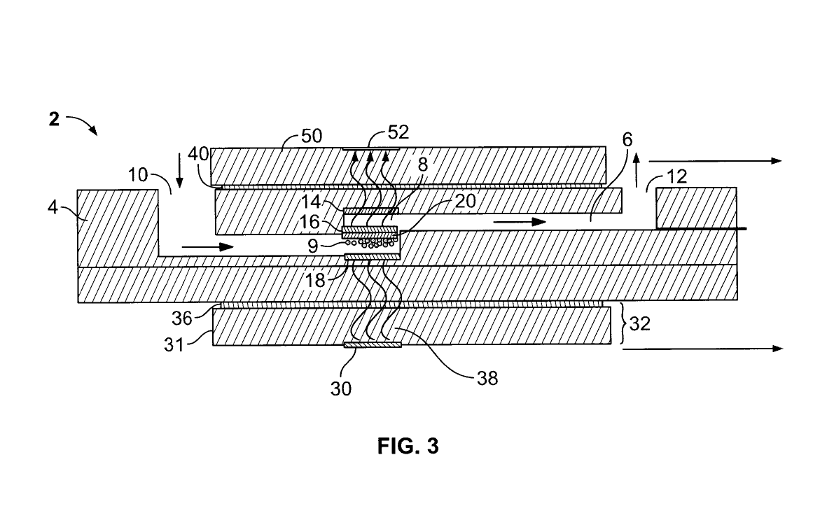

Referring now to FIG. 3, therein illustrated is a side section view of one exemplary embodiment of the apparatus 2. For example, each microfluidic channel 6 can further define a microfluidic chamber 8. In the example of FIG. 3, the microfluidic channel 6 defines a microfluidic chamber 8. The microfluidic chamber 8 can be a cavity within the microfluidic channel 8 having a greater cross-sectional area than other portions of the microfluidic channel 6.

Referring now to FIGS. 1, 2, and 3 microorganism or biological material 9 is received within the at least one microfluidic channels 6 of the chip 4. For example, the microorganism or biological material 9 can comprise at least one type of photosynthetic microorganism that undergoes photosynthesis when exposed to light in certain spectral ranges. Water sample of the water for which the pollution level is to be determined can also be received in the at least one microfluidic channels 6. For example, the water sample can be water polluted with chemical pollutant, organic or inorganic, like herbicides or other toxic substances. For example, the water sample can be collected from water drained from farmlands.

For example, the microorganism or biological material 9 and the water sample received in the microfluidic channel 6 can be mixed in the microfluidic channel 6 to form a composition. The can be mixed previously, before being introduced in the channel. Properties of the composition comprising the microorganism or biological material 9 and the water sample in each of the microfluidic channels 6 can then be determined.

For example, according to exemplary embodiments of FIG. 3, microorganism or biological material and the water sample received in the microfluidic channel 6 can accumulate at the microfluidic chamber 8 and then collapse or group together in the chamber to form the composition.

Referring back to FIGS. 1, 2, and 3, for example, each microfluidic channels 6 can further define a first opening 10 at a first end of the microfluidic channel 6 and a second opening 12 at a second end of the microfluidic channel 6.

For example, according to FIG. 3, microfluidic chamber 8 of each microfluidic channels 6 are in fluid communication with outside space through both the first opening 10 and the second opening 12.

For example, the microorganism or biological material 9 can be first inserted, or pre-inserted during fabrication of the chip, into the microfluidic channel 6. The chip 4 can then be submerged into a volume of water for which the level of pollution is to be determined. The chip 4 is submerged such that at least one of the first opening 10 or second opening 12 is in communication with the volume of water. A sample of the volume of water then enters either the first opening 10 or second opening 12, or both, to be received in the microfluidic channel 6.

For example, at least two electrodes 14 (see FIG. 3) can be positioned in each of the at least one microfluidic channels 6 of the chip 4. The at least two electrodes are each connected to an electric detector for detecting at least one electrical property of the composition received in each of the microfluidic channels 6. Additional electrodes can be positioned in the microfluidic channel to permit a greater number of electrical properties to be detected. For example, the electric detector can cause a DC or an AC current to be emitted between the at least two electrodes. For example, the electric detector can be configured to detect at least one of the following properties of the composition, such as resistivity, conductance, pH levels, temperature and turbidity of a liquid.

For example, according to FIG. 3, where the microfluidic channel 6 define a microfluidic chamber 8, electrodes 14, 16 and 18 of the electric detector can be positioned within the microfluidic chamber 8. For example, FIG. 3 shows three electrodes 14, 16 and 18, with electrode 14 positioned in a top portion of the microfluidic chamber 8, porous electrode 16 positioned in an intermediate portion of the microfluidic chamber 8 and electrode 18 positioned in a bottom portion of the microfluidic chamber 8. Electrode 16 is in contact with the filter 20, where electrode 16 could be above or below filter 20. Electrode 16 allows passage of water therethrough. For example, the three electrodes can comprise one working electrode (WE), one counter electrode (CE) and one reference electrode (REF).

Continuing with FIG. 3 for example, each of the microfluidic chambers 8 can comprise a filter 20 for filtering the composition received in the microfluidic channels 6. The filter 20 can be at least semi-transparent. It can also be substantially transparent. In particular, the filter 20 is adapted to substantially restrict the flow of microorganism or biological material 9, of the composition through the microfluidic channel 6 and microfluidic chamber 8, while permitting flow of the water sample therethrough. For example, movement of the chip 4 causes flow of the composition back and forth within the microfluidic channel 6. It will be appreciated that as the composition is filtered by the filter 20, an amount of a plurality of a microorganism or biological material 9 will be collected at the filter 20. For example, the filter 20 is semi-transparent or substantially transparent to allow passage of a substantial amount of light through it. For example, the filter 20 can be a porous membrane having pores with diameters in a range between of about 0.05 um to about 10 um. For example, the filter 20 can be formed of a suitable polymer, such as PET, PEN, PS, or Teflon, of alumina, glass or cellulose.

In some exemplary embodiments, at least one of the electrodes is connected to the filter 20 (see FIG. 3). In such embodiments, the electrode can be semi-transparent or substantially transparent to allow light to pass through it. For example, the electrode 14 can comprise a nanomaterial including plurality of members defining a plurality of pores for allowing passage of light and water therethrough. The nanomaterial can be conductive and can have a diameter in the range of the nanometer. The nanomaterials associated with the filter can be interweaved to define a plurality of porous openings having width/area in the range of about 0.05 to about 10 μm. The water sample can pass through the porous openings Additionally, a substantial amount of light can pass through the porous openings or be transmitted by the nanomaterial. For example, the nanomaterial comprised in the electrode 14 can be in the form of nanotubes, nanofilaments, nanowires, nanorods etc. The nanomaterial can be carbon, silver, platinum, nickel, copper, gold or other suitable metals, alloys or derivatives thereof. For example, the nanomaterial can comprise carbon nanotubes, including single-walled or multi-walled carbon nanotubes. For example, the nanomaterials can be graphene, a mixture of nanowires and carbon nanotubes or composite nanowire formed from a mixture of metals. For example, the conductive nanomaterials can have a resistance below microorganism or biological material. Referring back to FIGS. 1, 2 and 3, for example, apparatus 2 for evaluating water pollution can comprise at least one light source 30. For example the light source 30 can be supported by a substrate 31 within an illuminating layer 32 that can be planar. The light source 30 can be horizontally arranged, for example within a same plane defined by the illuminating layer 32 such that light is emitted at various locations from the illuminating layer 32.

For example the at least one light source 30 can be at least one organic light emitting diodes (OLEDs). Organic light emitting diodes can have a miniature size, thereby allowing the illuminating layer to have a very thin profile. However, it is contemplated that other types of light sources being miniature in size can be used. Such light sources are intended to be covered by the present description.

For example, the chip 4 can include microlenses to focus the emission light from the light source 30. For example, microlenses can be included into the light layer 32 or into the light filtering layer 36.

For example, light emitted by the light source 30 can have specific spectral properties. The light emitted by the light source 30 can cause certain reactions to the microorganism or biological material 9 received within the microfluidic channel 6 and/or microfluidic chamber 8.

In particular, for example, where the microorganism or biological material 9 comprises at least one type of photosynthetic microorganism, exposing the at least one type of photosynthetic microorganism to the light emitted from light source 30 causes it to absorb the light and undergo photosynthesis. Absorption of light by the at least one type of photosynthetic microorganism is due to its chlorophylls and its pigments (for example carotenoids, phycocyanins and phycoerythrins). Absorbed photons are used to perform photosynthesis. Any excess energy not used for photosynthesis is remitted as heat or fluorescent light. Causing the at least one type of photosynthetic microorganism to undergo photosynthesis and emit excess energy as fluorescent light will herein be referred to as “exciting” the photosynthetic microorganisms. Light emitted from the light source 30 for exciting the at least one type of photosynthetic microorganism will herein be referred to as “excitation” light.

For example, excitation light emitted from the light source 30 includes emitted photons having wavelengths in a spectral range corresponding to the spectral range wherein the received photosynthetic microorganisms are excited.

For example at least one first optical filter 36, which can form a filtering sub-layer of the illuminating layer 32 and is positioned between the substrate 31 supporting the light source 30 and the chip 4 to filter light emitted from the light source 30. Accordingly the light emitted by the at least one light source 30 having known spectral properties are filtered by the optical filter such that excitation light emitted from the top surface of the illuminating layer 32 has specific spectral properties for causing reaction in the microorganism or biological material 9.

For example, the optical filters 36 can exhibit limited auto-fluorescence, high transmittance at the desired spectral range, high attenuation in the unwanted spectral range, and is inexpensive to fabricate. For example, optical filter 36 can be fabricated as a dye-doped resin. For example, the optical filter 36 can be dichroic, absorbing, or polarizing.

For example, the at least one light source 30 can be selected or configured to directly produce light having specific spectral properties for causing the microorganism or biological material 9 to be excited. For example, where the at least one light source 30 is an OLED, excitation light having specific spectral properties for exciting the microorganism or biological material 9 can be emitted by appropriately selecting the organic emissive layers of the OLED. Alternatively excitation light having specific spectral properties for exciting the photosynthetic microorganisms can be emitted by varying the intensities of differently coloured OLED an array of OLED and/or different emission wavelength OLED. It will be appreciated that where the at least one light source 30 directly produces excitation light having desired specific spectral properties, it can be not necessary to have at least one optical filter 36 within the illuminating layer 32.

According to some embodiments, a single light source 30 can be used to emit light to the microfluidic channels, and microfluidic chambers, of the chip 4. For example, FIG. 2 shows one light source 30 emitting light over a portion of the length of the channel 6.

Referring now to FIG. 3, for example, to allow maximum exposure of microfluidic chamber 8 to light from the at least one light source 30, the chip 4 and the at least one light source 30 can be positioned such that at least some of the at least one microfluidic chamber 8 is substantially aligned with one of the light source 30 in a direction transverse to the plane defined by the chip 4. For example, at least one microfluidic chamber 8 can be aligned with the at least one light source 30 in a direction orthogonal to the plane defined by the chip 4.

For example, the filter 20 of the microfluidic chamber 8 can also be positioned within the microfluidic chamber 8 to receive maximum exposure to light from the at least one light source 30. For example, the filter 20 can also be positioned such that the filter 20 of at least one of microfluidic chamber 8 can be substantially aligned with the at least one light source 30 in a direction transverse to the plane defined by the chip 4. For example, the at least one microfluidic chamber 8 can be aligned with the at least one light source 30 in a direction orthogonal to the plane defined by the chip 4.

For example, to further increase exposure of the filter 20 to light from the at least one light source 30, where the filter 20 has a planar shape, the filter 20 can be positioned to be parallel to the chip plane and transverse the direction of the light emitted from the at least one light source 30. In the exemplary embodiment of FIG. 3, the filter 20 is positioned horizontally within the microfluidic chamber 8 and in parallel with the chip plane. It will be appreciated that since the filter 20 substantially restricts the flow of microorganism or biological material 9 such that the microorganism or biological material 9 is collected at the filter 20 according to this positioning, a large quantity of the members of the microorganism or biological material 9 are exposed to the light from the at least one light source 30.

For example in FIG. 3, photons 38 being represented by waves are emitted by the at least one light source 30. The at least one light source 30 is positioned in a plane defined by the illuminating layer 32 to be aligned with the chamber 8 in a direction transverse to the planed defined by the chip 4. Photons 38 in the emitted excitation light are absorbed by the microorganism or biological material 9 accumulated at the filter 20 of the microfluidic chamber 8, causing the microorganism or biological material 9 to react. In particular, where the microorganism or biological material 9 is the at least one photosynthetic microorganism, photons 38 within a specific spectral range will cause the microorganism or biological material 9 to be excited.

For example, the substrate of chip 4 can be fabricated to be semi-transparent or substantially transparent at bottom surface 28. For example, the substrate of chip 4 can be semi-transparent or substantially transparent at the locations of some of the microfluidic chambers 8. This restricts each microfluidic chamber 8 from being exposed to excitation light from a non-aligned light source 30. For example, chip 4 can be formed to be semi-transparent or substantially transparent to allow light emitted upwardly from the microfluidic channels 6 and/or microfluidic chambers 8 to reach other layers disposed above the chip 4.

For example chip 4 can be formed to be substantially opaque in an upper and in a lower portion of the chip 4 except for the at least one transparent gap. For example chip 4 can comprise a substantially opaque sub-layer 39 defining the at least one transparent gaps. Light emitted from a the microfluidic chambers 8 after having have been exposed to excitation light emitted from the illuminating layer 32 can have varying spectral properties that can depend on the properties of the microorganism or biological material and/or water received in the microfluidic chamber 8. To restrict mixing of light emitted from different microfluidic chamber 8, the chip 4 can be fabricated to be semi-transparent or substantially transparent at top surface only at the locations of each of microfluidic chambers.

For example the apparatus 2 can comprise at least one second optical filter 40, which can form a filtering layer. For example, the filtering layer can be supported by the chip 4.

For example, the at least one second optical filter 40 can have a longpass or a passband corresponding to the spectral range of fluorescent light emitted by the excited photosynthetic microorganisms received in the chip 4. For example, light emitted from the chip 4 can comprise a mixture of excitation light emitted from the at least one light source 30 not absorbed by the photosynthetic microorganisms and fluorescent light emitted from the plurality of photosynthetic microorganisms received in the chip 4. When such light is filtered by the at least one optical filter, light in the fluorescent light spectral range is transmitted while light outside this spectral range, for example excitation light from the illuminating layer 32 not absorbed, is attenuated.

For example, the optical filter 40 exhibits limited auto-fluorescence, high transmittance at the desired spectral range, high attenuation in the unwanted spectral range, and is inexpensive to fabricate. For example, the optical filter can be fabricated as a dye-doped resin. For example, the optical filters 40 can be dichroic, absorbing, or polarizing.

For example the apparatus 2 can comprise the at least one photodetector 52. For example, the at least one photodetector 52 can be any type of detector that determines the intensity of photons in light emitted from the chip 4 and being filtered by optical filters 40 where such optical filters 40 are used. The at least one photodetector 52 can be supported on a semi-transparent or substantially transparent substrate 50.

For example, the at least one photodetector 52 can be organic photodetector. For example, the organic photodetector can be fabricated using semiconducting polymers with alternating thieno[-3,4-b]-thiophene and benzodithiophene or with phthalocyanin organic material and other semiconducting material that absorbs at the desired wavelength.

For example, the at least one photodetector 52 can be inorganic, such as being formed of silicon.

For example, the at least one photodetector 52 can detect an intensity level of photons received by the at least one photodetector 52 and return an amplitude value, such as voltage or power value.

For example, the at least one photodetector 52 can be an image sensor, such as a CCD or CMOS, sensor that returns electronic signal for the light sensed. For example the electronic signal can be a frequency response of the detected light.

For example, the at least one photodetector 52 can be any light detector that can detect properties of light emitted from the chip 4 that are in a spectral range corresponding to the spectral range of fluorescent light emitted by the excited photosynthetic microorganisms in the microfluidic channels. For example, the at least one photodetectors 52 can be optimized for detecting light in this spectral range.

Referring back to FIG. 3, for example, the at least one photodetector 52 can be positioned to be substantially aligned with one of the microfluidic chambers 8. For example, the at least one photodetector 52 can be aligned with the at least one microfluidic chamber 8 in a direction transverse to the planed defined by the chip 4. For example, the at least one photodetector 52, the at least one microfluidic chamber 8 and the at least one light source 30 can be aligned in a direction orthogonal to the plane defined by the chip 4.

For example, the at least one photodetector 52 can be positioned to be further substantially aligned with the filter 20 of the at least one microfluidic chamber 8.

In some exemplary embodiments, the at least one light source 30 is not necessarily aligned with the at least one microfluidic chamber 8 and the at least one light source 30 can emit light into more than one microfluidic chamber 8. For example, this can be the case where the at least one light source 30 is an OLED, which has a very high index of refraction and wide angle of emission. However, in some exemplary embodiments, as illustrated in FIG. 3, the at least one light source 30 can be aligned with the photodetector 52 and the microfluidic chamber 8 that are already aligned together.

As described above, in some exemplary embodiments, more than one light source 30 can be aligned with one photodetector 52 and one microfluidic chamber 8 that are already aligned together. Furthermore, each of the light sources 30 that are aligned can emit light in a different spectral range.

For example, in FIG. 3, photons 38 are shown being emitted from the at least one light source 30 in a direction transverse to the chip plane. The photons travel to the aligned microfluidic chamber 8 of the microfluidic channel 6 to expose the microorganism or biological material 9 received therein. The filter 20 is positioned in the at least one microfluidic chamber 8 in alignment with the microfluidic at least one chamber 8 and the at least one light source 30. As the members defining the at least one microorganism or biological material 9 are collected at the filter 20, the members defining the microorganism or biological material 9 are also exposed to the light from the at least one light source 30. When the filter 20 is semi-transparent or substantially transparent, light from the at least one light source 30 passes through the filter 20 towards the at least one photodetector 52. Additionally, fluorescent light emitted from the members defining the microorganism or biological material 9 as they are excited also passes through the filter 20 towards the at least one photodetector 52. The at least one photodetector 52 being further aligned with the at least one microfluidic chamber 8 and the at least one light source 30 detects intensity of light from the microfluidic chamber 8. In particular, it detects intensity of light in the spectral range corresponding to the fluorescent light emitted by the microorganism or biological material. Furthermore, three electrodes 14, 16, and 18 can placed within the microfluidic chamber 8.

It will be appreciated that alignment of one photodetector, one microfluidic chamber and one light source in a direction transverse the chip plane in conjunction with placement of electrodes connected to the electric detector advantageously allows a plurality of measurements of properties to be taken of the composition in the same microfluidic chamber 8. For example, the level of fluorescent light that is emitted from the at least one microfluidic chamber 8 that is detected by the aligned at least one photodetector 52 allows for a determination of the amount, for example a concentration, of microorganisms in the composition. This provides a first indication of the pollution level of the water sample in the composition. For example, properties, for example conductance, of the composition that are measured by the electrodes and electric detector provide further indications of the pollution level of the water sample in the composition.

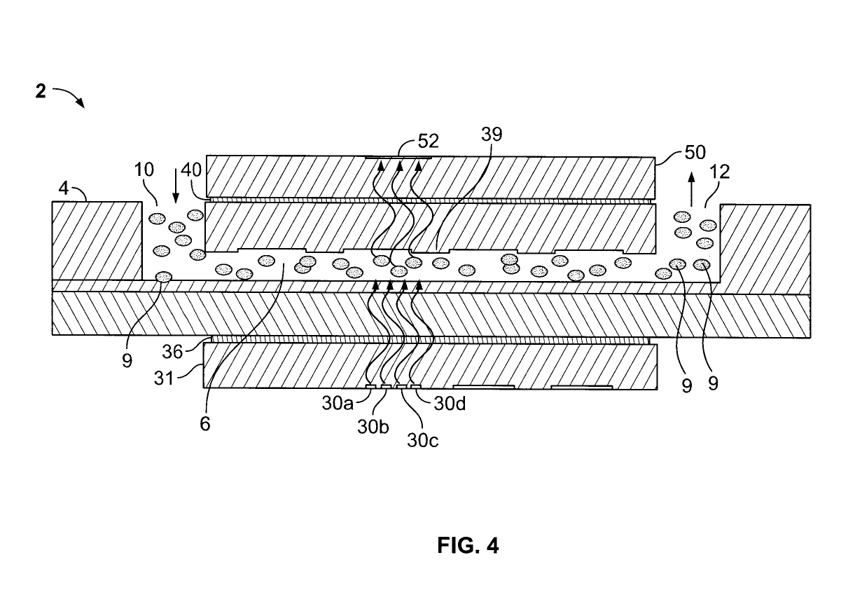

Referring now to FIG. 4, for example, a plurality of light sources 30a-30d can be aligned with a single microfluidic chamber 8. For example each of the light source 30a, 30b, 30c and 3d can be aligned with one microfluidic chamber 8 can emit light in a different spectral range. Where the at least one microorganism or biological material 9 is at least one type of photosynthetic microorganism, light in each of the spectral ranges can excite various pigments of the microorganisms that cause fluorescent light to be emitted. For example, some of the light can be in spectral ranges that excite pigments of the at least one type of microorganism other than the chlorophyll.

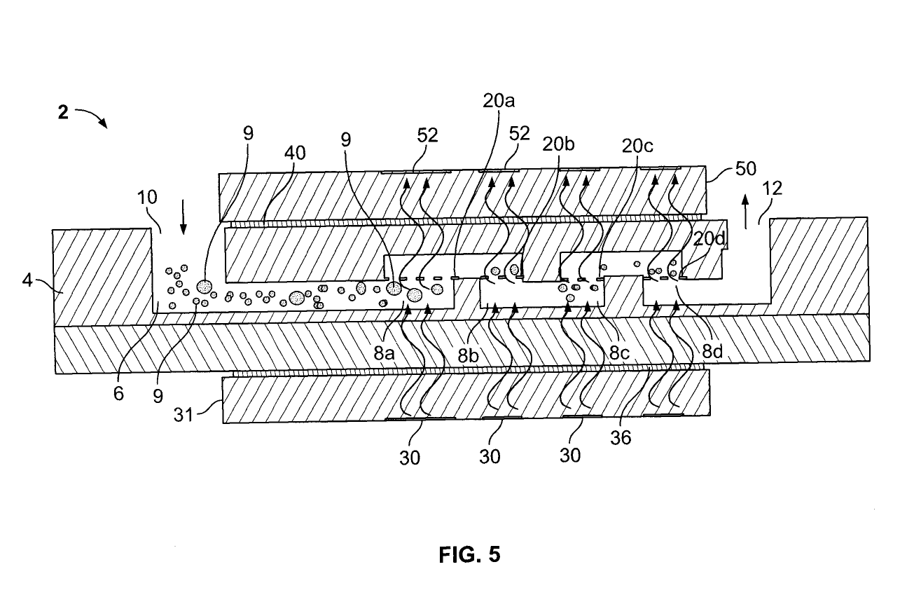

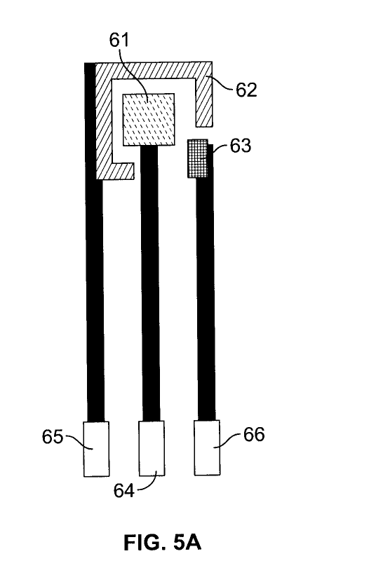

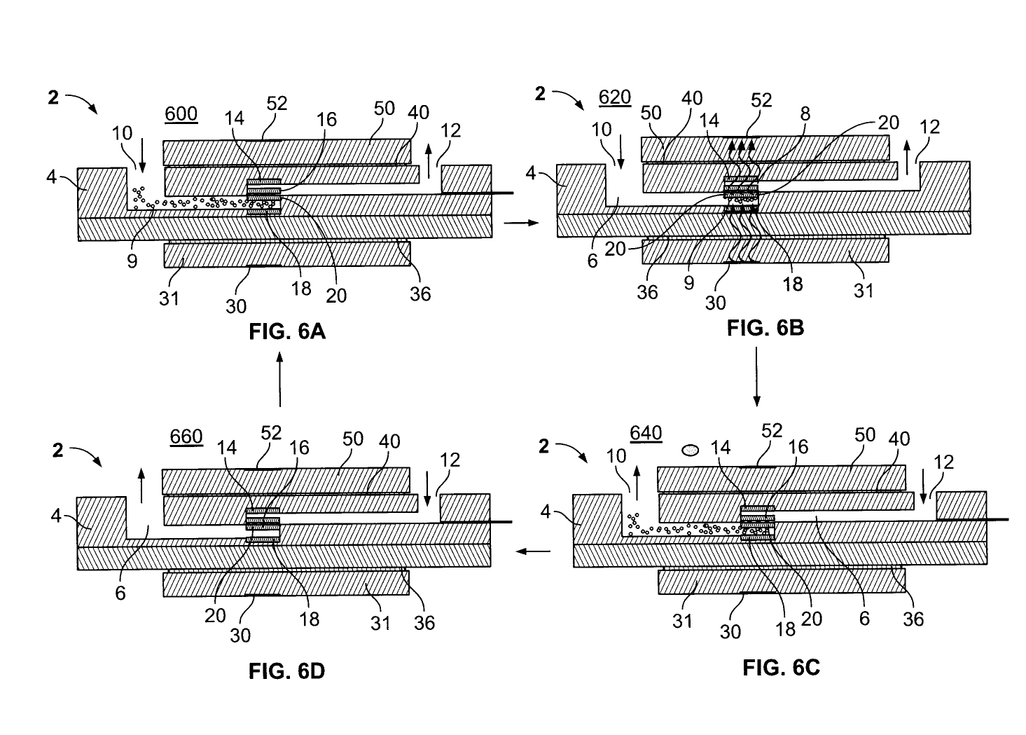

Referring now to FIG. 5, therein illustrated is a side view of some exemplary embodiments of the chip 4, wherein the at least one microfluidic channel 6 defines more than one microfluidic chambers 8. For example, one microfluidic channel 6 comprises microfluidic chambers 8a, 8b, 8c and 8d. Each microfluidic chamber can further have a filter. For example, microfluidic chambers 8a, 8b, 8c and 8d respectively have filters 20a, 20b, 20c and 20d. For example, the porous openings of the filters 20a, 20b, 20c and 20d can become progressively smaller in the direction from first opening 10 towards second opening 12. It will be appreciated that the filter 20a will only restrict flow of members of the at least one microorganism or biological material 9, with smaller members of microorganism or biological material 9 passing through the filter 20a. As a result, the members of the at least one microorganism or biological material 9 found in each of microfluidic chambers 8a, 8b, 8c and 8d will have different sizes. Separating the members of microorganism or biological material 9 in this manner allows for separately measuring of members of the at least one microorganism or biological material 9 of different sizes. A single type microorganism or biological material 9 can be used. It should be noted that like in FIGS. 3 and 6A-6C, the filter 20a, 20b and 20c can be connected to electrodes. In fact, electrodes can be connected to the filters (below or above) and thus provide an electric detector. The electrodes can be porous and they can comprise at least one nanometarial. These electrodes can be disposed one beside the other and/or one above the other.

Referring now to FIG. 5A, therein illustrated is a plan view of a planar electrical detector 60 having electrodes that are coplanar. The planar electrical detector 60 has a three-electrode configuration formed of a working electrode 61, a counter electrode 62, and a reference electrode 63. The working electrode 61 is connected to a first lead 64. The counter electrode 62 is connected to a second lead 65. The reference electrode 66 is connected to a third lead 66.

According to various exemplary embodiments, the planar electrical detector 60 is positioned within the microfluidic chamber 8. For example, the electrical detector 60 is positioned such that the plane defined by the co-planar working electrode 61, counter electrode 62, and reference electrode 63 is substantially parallel with the plane of the chip 4.

According to various exemplary embodiments, at least the working electrode 61 is semi-transparent. The semi-transparency of the working electrode 61 allows light emitted from the light source 30 to pass through the working electrode 61 and reach the photodetector 52. For example, the working electrode 61 can also be porous. The working electrode 61 being porous allows liquid found in the microfluidic channel 6 and/or the microfluidic chamber 8 to flow through the working electrode 61.

According to various exemplary embodiments, the working electrode 61 is positioned within the microfluidic chamber 8 to be substantially aligned with one of the light sources 30 in a direction transverse to the plane defined by the chip 4. For example, at least the working electrode 61 can be aligned with the at least one light source 30 in a direction orthogonal to the plane defined by the chip 4. Alignment of the working electrode 61 with the light source 30 positions the electrode 61 with a location where the microorganism or biological material will most likely undergo photoactivity. For example, at least the working electrode 61 is positioned proximate the filter where microorganisms or biological material received in the microfluidic chamber are entrapped.

According to various exemplary embodiments, the counter electrode 62 and the reference electrode 63 are semi-transparent. The semi-transparency of the counter electrode 62 and the reference electrode 63 allow light emitted from the light source 30 to pass through the counter electrode 62 and the reference electrode 63 and reach the photodetector 52. For example, the counter electrode 62 and the reference electrode 63 can also be porous. The counter electrode 62 and the reference electrode 63 being porous allows liquid found in the microfluidic channel 6 and/or the microfluidic chamber 8 to flow through the working electrode 61.

According to various exemplary embodiments, the counter electrode 62 and the reference electrode 63 is positioned within the microfluidic chamber 8 to be substantially aligned with one of the light source 30 in a direction transverse to the plane defined by the chip 4. For example, the counter electrode 62 and the reference electrode 63 can be aligned with the at least one light source 30 in a direction orthogonal to the plane defined by the chip 4. Alignment of the counter electrode 62 and the reference electrode 63 with the light source 30 positions the electrodes 62 and 63 with a location where the microorganism or biological material will most likely undergo photoactivity. For example, at least the counter electrode 62 and the reference electrode 63 is positioned proximate the filter where microorganisms or biological material received in the microfluidic chamber are entrapped.

According to various exemplary embodiments, the working electrode 61, the counter electrode 62, and the reference electrode 63 are formed of a plurality of nanomaterial members defining a plurality of pores. The nanomaterial can be conductive and can have a diameter in the range of the nanometer. The nanomaterials associated can be interweaved to define a plurality of pores. For example, the nanomaterial can be in the form of nanotubes, nanofilaments, nanowires, nanorods etc. The nanomaterial can be carbon, silver, platinum, copper, or other suitable metals, alloys or derivatives thereof. For example, the nanomaterial can comprise carbon nanotubes, including single-walled or multi-walled carbon nanotubes. For example, the nanomaterials can be graphene, a mixture of nanowires and carbon nanotubes or composite nanowire formed from a mixture of metals. For example, the conductive nanomaterials can have a resistance below microorganism or biological material.

According to one exemplary embodiment, each of the working electrode 61, counter electrode 62 and reference electrode 63 are formed of silver nanofilaments. For example, the silver nanofilaments forming at least two of the working electrode 61, counter electrode 62 and reference electrode 63 are coated with platinum. It has been found that platinum coating increases electrical and chemical efficiency as well as chemical stability with the environment containing algae. For example, nanofilaments forming the working electrode 61 and nanofilaments forming the counter electrode 62 are coated with platinum. For example, the reference electrode 63 is left bare.

According to various exemplary embodiments, the electrical detector can determine an oxygen concentration in the microfluidic chamber. For example, one or more of the electrodes can measure an electrical property that is indicative of an oxygen concentration in the microfluidic chamber.

For example, at least one of the illuminating layer 32, chip 4, substrate 31 and substrate 50 of the apparatus 2 can be made to be thin such that the apparatus 2 can have a miniature size. The volume of the detection chamber can range from a few microliter to several hundred microliter. For example about 1 μL to about 500 μL, about 5 μL to about 400 μL, about 10 μL to about 250 μL, about 5 μL to about 150 μL, about 100 μL to about 300 μL, about 10 to about 100 μL For example, it will be appreciated that the at least one light source 30 can also be made to have a miniature size. For example, OLEDs are miniature the at least one light source 30 that can be supported by a thin substrate.

The miniature size of the apparatus 2 according to various embodiments described herein, allows it to be portable. Unlike laboratory techniques that require cumbersome equipment, the miniature size of the apparatus 2 allows it to be easily deployed in the field.

The ease of fabrication and the use of readily available components allow the apparatus 2 according to various embodiments described herein to be inexpensive to manufacturer. For example, it is contemplated that the apparatus 2 can be portable and disposable. Alternatively, at least one sub-components of the apparatus 2 can be replaceable or disposable. For example, the chip 4 comprising the at least one microfluidic channel 6 can be replaced between uses. Moreover, once measurements are taken, the chip 4 can be disposed of and new chip 4 can be inserted into the apparatus 2 for evaluating pollution of further samples of water.

For example, that apparatus 2 can further comprise at least one input-output port for connecting the apparatus 2 to an external device. For example, the apparatus 2 can receive control signals from the external device through the input-output port for controlling the at least one light source 30 to emit a light, for controlling the at least one electrode 14, 16 or 18 to make a measurement of electrical property, and/or for controlling the at least one photodetector 52 for detecting a light. For example, the external device can have a controller, such as control module, that sends the control signals to the apparatus 2.

For example, the apparatus 2 can comprise a controller implemented on-board the apparatus 2. In such a case, the on-board controller controls the light source 30, the at least one electrode 14, 16 and 18 and/or the at least one photodetector 52.

The controller of the apparatus 2 or of the external device described herein can be implemented in hardware or software, or a combination of both. It can be implemented on a programmable processing device, such as a microprocessor or microcontroller, Central Processing Unit (CPU), Digital Signal Processor (DSP), Field Programmable Gate Array (FPGA), general purpose processor, and the like. In some embodiments, the programmable processing device can be coupled to program memory, which stores instructions used to program the programmable processing device to execute the controller. The program memory can include non-transitory storage media, both volatile and non-volatile, including but not limited to, random access memory (RAM), dynamic random access memory (DRAM), static random access memory (SRAM), read-only memory (ROM), programmable read-only memory (PROM), erasable programmable read-only memory (EPROM), electrically erasable programmable read-only memory (EEPROM), flash memory, magnetic media, and optical media.

For example, the apparatus 2 can further comprise an on-board memory for storing measurements taken by the at least one electrode and the at least one electric detector and/or by the at least one photodetector 52. For example, the memory can be any suitable memory such as flash memory, magnetic media, or optical media.

For example, the apparatus 2 can further comprises a power source, such as a battery, solar cells for powering the controller and the memory. For example, where the apparatus 2 comprises the on-board controller, memory, and power source, apparatus 2 can be used autonomously without having to be connected to an external device. In such a case, the apparatus 2 can be used in the field for evaluating various water sources on its own. Obtained measurements can be saved in the on-board memory. The apparatus 2 can be connected to an external device through the input-output port to download the obtained measurements to the external device.

For example, a method for evaluating the pollution level of a water sample comprises mixing a plurality of at least one type of microorganism or biological material s, which can be at least one type of photosynthetic microorganisms with the water sample.

For example, the at least one microorganism or biological material 9, which can be or not mixed in a liquid. It can be first inserted into the at least one microfluidic channel 6 of a chip 4. For example, prior to inserting the water sample, multiple liquid mixtures containing the microorganism or biological material 9 can be inserted, each mixture being inserted into different channels 6 of the chip 4. For example, each microfluidic channel 6 can be inserted with a different type of microorganism or biological material, such as different types photosynthetic microorganisms. For example, each microfluidic channel 6 can be inserted with liquid mixture having a different concentration of a type of microorganism or biological material. Alternatively, various types of microorganism or biological material s and various concentrations of microorganism or biological material s can be inserted into the various microfluidic channels 6 of the chip 4.

For example, the water sample can be directly injected alone in the chip 4 before the measurement. For example, the water sample can be filtered before to be mixed with the at least one type of microorganism or biological material and then injected in the chip 4. For example, the water sample can be filtered before to be mixed with the at least one microorganism or biological material, filtered again to only get the at least one microorganism or biological material. The filtered composition is injected in the chip 4 to do the measurement.

The at least one microorganism or biological material 9, can be inserted as an aqueous composition in the at least one channel 6 and then the water sample can be inserted therein. Both the at least one microorganism or biological material 9 and the water sample can be mixed together so as to obtain a composition and then, the composition is inserted in the at least one channel 6. Alternatively, the water sample can be introduced into the at least one channel 6 and then, the at least one microorganism or biological material 9 is introduced (as is or in an aqueous composition).

For example, the at least one microorganism or biological material 9 can be pre-inserted into the microfluidic channels 6 of the chip 4 during fabrication. The chip 4 can then be stored to be later used for detecting a level of pollution of a water sample.

Insertion of various types and/or concentrations of microorganism or biological material into the at least one microfluidic channel 6 allow evaluation of water samples having different level of pollutants or different types of pollutants. For example, different types or different concentrations of microorganism or biological material can be better suited for accurately measuring a water sample having a certain level of pollution or certain type of pollution. By injecting various types of microorganism or biological material and/or various concentrations of microorganism or biological material into the various microfluidic channels 6 of the chip 4 of a single apparatus 2, the single apparatus 2 can be used to accurately evaluate pollution for various water samples having a wide range of properties. It can also better evaluate the presence of various pollutants in the water sample.

Where the at least one microorganism or biological material 9 is at least one photosynthetic microorganism, some relevant properties of the at least one photosynthetic microorganism are known. For example, the spectral range of light that causes the photosynthetic microorganisms to be excited can be known. The spectral range of fluorescent light emitted by the photosynthetic microorganisms as excess energy when undergoing photosynthesis can also be known. The rate of decay of the activity of photosynthetic microorganisms for various levels of water pollution can also be known.

For example various types of photosynthetic microorganisms can be mixed with the water sample. For example, the type of photosynthetic microorganism can be selected depending on the known properties of the type of photosynthetic microorganism and the anticipated quantity and/or type of pollutants in the water sample. For example, the at least one photosynthetic microorganism can be microalgae, bacteria, cyanobacteria, and other living organisms that produce pigments. When a photosynthetic activity is measured, the at least one photosynthetic microorganism can be, for example microalgae, cyanobacteria, or photosynthetic bacteria.

For example, the at least one photosynthetic microorganism can be provided in a liquid mixture or an aqueous composition having a known concentration of photosynthetic microorganisms. For example, a known quantity of liquid mixture or composition of photosynthetic microorganisms can be mixed with the water sample by inserting the composition and the water sample into one of the at least one microfluidic channel 6 of the chip 4 of any one of the exemplary embodiments of the apparatus 2 described herein. Accordingly, the quantity of photosynthesis microorganisms can also be known.

For example, after insertion of the at least one microorganism or biological material 9 in the at least one microfluidic channel 6, first measurements can be taken to obtain control measurements. At this point, the members of the at least one microorganism or biological material 9 should still all be in a health state having not yet been exposed to a water sample having a certain pollution level. Therefore, the control measurements should offer a useful point of reference.

For example, control measurement can be obtained by detecting at least one electrical property of the healthy members of the at least one microorganism or biological material 9 in the at least one microfluidic channel 6 using the electrodes 14, 16 and 18 placed therein. Furthermore, where the at least one microorganism or biological material is at least one photosynthetic microorganism, light can be emitted into the at least one microfluidic channel 6 to excite the microorganisms, and a first level of light emitted from the at least one channel 6 can be detected to obtain a control fluorescence measurement.

For example, after insertion of the at least one microorganism or biological material 9, into the at least one microfluidic channel 6, the water sample can be inserted into each of the microfluidic channels 6. Water sample can be collected by submerging the first opening 10 into a volume of water to be evaluated for pollution level. For example, the volume of water can be water drained from farmlands where herbicide has been used. A sample of the volume of water is received into each of the at least one microfluidic channel 6 through either one, or both of the first opening 10 or second opening 12 of each of the at least one microfluidic channel 6. The water sample and the at least one microorganism or biological material 9 are inserted in the at least one microfluidic channel 6 to form a composition.

For example, where the at least one microfluidic channel 6 defines a microfluidic chamber, the at least one microorganism or biological material 9 and the water sample can be mixed in the at least one microfluidic chamber 8. For example, where the filter 20 is further positioned within the at least one microfluidic chamber 8, the composition can be filtered through the filter 20 such that the at least one microorganism or biological material 9 is collected at the filter.

After mixing the at least one microorganism or biological material 9 with the water sample, the at least one microorganism or biological material 9 can react to pollutants in the water sample. For example, pollutants in the water sample can cause decay of the photosynthetic activity of the at least one microorganism or biological material 9. The amplitude and rate of decay can vary according to the level of pollution in the water sample. Therefore, the decay of the activity of the microorganism or biological material 9 provides an indication of the level of pollution.

For example, a waiting time can be allowed to pass after mixing the at least one microorganism or biological material 9 and the water sample to allow the at least one microorganism or biological material 9 to sufficiently reacts to pollutants in the water sample. The waiting time is dependent of the type of pollutants present in the water sample

For example, excitation light is emitted onto the composition comprising the water sample and the at least one microorganism or biological material 9 to excite them. For example, the excitation light is emitted only after the waiting time for allowing the at least one microorganism or biological material 9 to sufficiently react to pollutants in the water sample has expired.

For example, the at least one light source 30 of the apparatus 2 described herein emits excitation light onto at least one of the composition received in at least one of the microfluidic channel 6. For example, where the microfluidic channels 6 each define a microfluidic chamber 8, each light source 30 can emit light onto the microfluidic chamber 8 that is aligned with it in a direction transverse to the plane defined by the chip 4.

For example, when emitting excitation light from the at least one light source 30 onto a composition that comprises the at least one type of photosynthetic microorganism, the emitted light can have wavelengths corresponding to the spectral range causing the at least one microorganism to undergo photosynthesis and emit excess energy absorbed from the light as fluorescent light. Alternatively, light emitted by the at least one light source 30 can be filtered by at least one optical filters such that light exposing the at least one type of photosynthetic microorganism to have a spectral corresponding to the spectral range wherein the at least one type of photosynthetic microorganism is excited.

For example, where the at least one type of photosynthetic microorganism can be green algae, such as Chlamydomonas reinhardii, the excitation light emitted can have a spectral range within approximately 400-500 nm. For example, the at least one type of photosynthetic microorganism can be green algae, diatoms, cryptophytes, red algae etc.

For example, fluorescent light emitted by the at least one type of photosynthetic microorganism can be detected. For example, the level of fluorescent light can be detected as a measure of energy or voltage of the light detected. For example, the level of fluorescent light can be detected as a frequency response of the light detected, the frequency response including spectral information for the level of fluorescent light. For example, according to embodiments described herein, the fluorescent light emitted by the at least one type of photosynthetic microorganism received in the at least one microfluidic channel 6 after being exposed to light emitted are detected by the at least one photodetector 52.

For example, the level of fluorescent light can be periodically detected for a length of time after emitting excitation light onto the composition of the at least one type of photosynthetic microorganism and the water sample.

It will be appreciated that the level of fluorescent light can depend on the quantity of the at least one type of photosynthetic microorganism emitting the fluorescent light. The quantity of the at least one type of photosynthetic microorganisms emitting fluorescent light further depend on the initial quantity of the at least one type of photosynthetic microorganisms prior to mixture with the water sample and amount of decay of the activity of the at least one type of photosynthetic microorganism after exposure to pollutants in the water sample. Such decay further depends on the level of pollutants in the water sample. Therefore, it will be appreciated that the level of fluorescent light detected provides a reliable indicator of the level of pollutants in the water sample.

For example, where the at least one type of photosynthetic microorganism are green microalgae, excitation light that is dominant in a near infra-red range, such as within a spectral range of approximately 400-500 nm, can be emitted onto the microalgae to cause the microalgae to emit fluorescent light having wavelengths in the approximately 650-800 nm spectral range.

For example, prior to detecting the fluorescent light emitted from the mixture of the at least one type of photosynthetic microorganism and the water sample, the emitted light can be filtered using at least one optical filters have a passband corresponding to the wavelengths range of the fluorescent light emitted by the at least one type of photosynthetic microorganism. For example, light emitted from the chip 4 is filtered by at least one optical filter 40 of filtering layer. It will be appreciated that the filtering suppresses light in a spectral range outside the spectral range of the fluorescent light, For example, where the light filtered by at least one optical filters from the chip 4 comprises excitation light and fluorescent light emitted, the excitation light, which has a spectral range in the stopband of the optical filters, is suppressed. Therefore the light detected will only be light in the spectral range of fluorescent light. Detecting a level of this light provides an accurate representation of the level of fluorescent light emitted from the at least one type of photosynthetic microorganism. For example a simple amplitude measurement, such as voltage of the fight detected, provides an accurate representation of the level of the fluorescent light.

Alternatively, light emitted from the composition comprising the at least one type of photosynthetic microorganism and the water sample can be detected without being previously filtered. Accordingly the at least one photodetector 52 detecting the emitted light returns an electronic signal comprising a frequency response of the light detected. The frequency response of the electronic signal comprises spectral information for a broad spectral range. For example, the spectral range of corresponding to the fluorescent light within broad spectral range of the frequency response can be analyzed to determine the level of fluorescent light emitted by the at least one type of photosynthetic microorganisms.

For example, the light emitted from the composition comprising of the at least one type of photosynthetic microorganism and the water sample includes a mixing of fluorescent light emitted from the excited at least one type of photosynthetic microorganism and of excitation light emitted onto the mixture of the at least one type of photosynthetic microorganism and the water sample. For example, to distinguish between the microorganisms-emitted fluorescent light and the excitation light, the excitation light initially emitted onto the composition of the at least one type of photosynthetic microorganism and the water sample can be selected to be dominant within a spectral range that does not substantially overlap with the spectral range of the fluorescent light emitted by the at least one type of photosynthetic microorganism after being excited.

For example, where the at least two electrodes connected to an electric detector are placed within the at least one microfluidic channel, at least one electrical property of the composition containing the at least one type of microorganism or biological material and the water sample can be detected. The at least one electrical property measured provide additional indicators of a level of pollution of the water sample.

For example, measurements of the at least one electrical property of the composition can be taken periodically over an interval of time to monitor decay of the activity of microorganism or biological material over time.

For example, according to embodiments wherein the at least one light source 30, the at least one microfluidic chamber 8, the filter 20 of the at least one microfluidic chamber 8 and the at least one photodetector 52 are substantially aligned, for example in a direction transverse to the chip plane, a level of light from the aligned microfluidic chamber 8 can be detected by the at least one photodetector 52. Additionally, by placing electrodes 14, 16 and 18 connected to the at least one electric detector in the at least one microfluidic chamber, measurement of properties can be taken of composition in the same aligned microfluidic chamber. According to some examples, the detecting of the light emitted from the at least one microfluidic chamber 8 and the measuring of the at least one electrical property in the same microfluidic chamber can be carried out simultaneously, or substantially at the same time. It will be appreciated that obtaining multiple measurements of a sample of composition within the at least one microfluidic chamber 8 at substantially the same time allows for better analysis of the level of pollution of the water sample, especially where measurement of the at least one property can deviate or fluctuate over time.

Measurements taken of the composition provide information regarding the pollution level in the water sample. For example, the at least one measured electrical property provides a first set of indicators of the pollution level of the water sample and level of light detected by the at least one photodetector provides a second set of indicators of the pollution level of the water sample. For example, the at least one measured electrical property and detected level of light of the composition can be compared with the control measurements obtained from the healthy at least one type of microorganism or biological material 9 to obtain further information regarding the level of pollution of the water sample.

According to some embodiments, subsequent to detecting the level of light emitted from the composition and/or the at least one measuring of electrical property of the composition, the at least one microfluidic channel 6 can be cleaned to allow insertion of further batch of microorganism or biological material 9 members and a further water sample for evaluating water pollution in this further water sample.

For example, the at least one microfluidic channel 6 and the at least one microfluidic chamber 8 can be cleaned by flushing them with a washing agent. For example ethanol and/or water can be used for the flushing. For example, the flushing can be performed several times.

Referring now to FIGS. 6a to 6d, therein illustrated are four states of the chip 4 during use of the apparatus 2 and subsequent washing of the apparatus 2. Referring to FIG. 6a, at state 600, members of the at least one type of microorganism or biological material 9 and the water sample are inserted through first opening 10 of the at least one microfluidic channel 6.

Referring now to FIG. 6b, at state 620, the members of the at least one microorganism or biological material 9 are collected by the filter 20 and are most concentrated within the at least one microfluidic chamber 8. At least one electrical property can be measured and a level light emitted from the at least one microfluidic chamber 8 can be detected.

Referring now to FIG. 6c, at state 640, after having completed measurement, a cleaning agent can be inserted through the second opening. It will be appreciated that second opening 12 is located on the opposite side of the filter 20 relative to where the at least one type of microorganism or biological material 9 members are located within the at least one microfluidic chamber 8. As the cleaning agent flows through the at least one microfluidic chamber 6 and, more particularly through the filter 20, the members of the at least one type of microorganism or biological material 9 collected at the filter 20 are washed away. As the cleaning agent exists through the first opening 10, the members of the at least one type of microorganism or biological material 9 also exit through the first opening 10.

Referring now to FIG. 6d, for example, after washing the at least one microfluidic channel 6 with the cleaning agent, the at least one microfluidic channel 6 will be in a clean state 660 and is ready to receive some more of the at least one type of microorganism or biological material 9 and water sample to be tested for making further measurements of pollution level of the water sample.

For example, after having evaluated several water samples, buildup of residue within the at least one microfluidic channel can begin to affect accuracy of results. Accordingly the chip 4 of the apparatus 2 can be disposed of and a new chip 4 comprising at least one microfluidic channel 6 and at least one microfluidic chamber 8 that are clean can be used for evaluation of additional water samples.

For example, the after at least one evaluations of water samples, the apparatus 2 can be disposed and a new apparatus is used for evaluating further water samples.

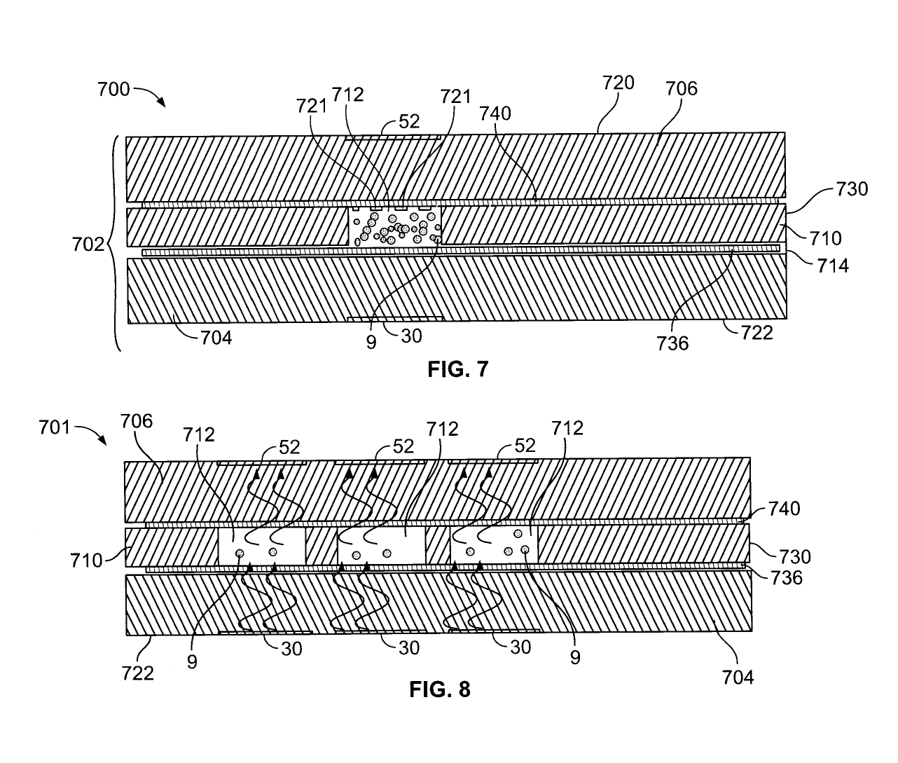

Referring now to FIG. 7, illustrated therein is a side section view of an apparatus 700 according to some exemplary embodiments having a slide 702 for evaluating a level of pollution of a water sample. For example, slide 702 can comprise a first substrate 704. The first substrate 704 can have at least one portion where it is semi-transparent or substantially transparent. For example substrate 704 can be similar to substrate 31 described herein with reference to FIGS. 2 to 6. The slide 702 can further comprise a second substrate. The second substrate 706 can also have at least one portion where it is semi-transparent or substantially transparent. For example, substrate 704 can be similar to substrate 50 described herein with reference to FIGS. 1 to 6. An intermediate layer 710 can be disposed between the first substrate 704 and the second substrate 706. For example, the intermediate layer 710 can be coupled to a surface of the first substrate 704 and a surface of the second substrate 706. For example, at least one of the first substrate 704 and the second substrate 706 can be substantially rigid to support the intermediate layer 710 and the other substrate. For example, the first substrate 704 or the second substrate 706 can be any suitable material that can be made to be semi-transparent or substantially transparent, such as glass.

When the intermediate layer 710 is disposed between the first substrate 704 and the second substrate 706, the two substrates are spaced apart and the ends of the two substrates define at least a first opening 714. For example, where the two substrates have corresponding quadrilateral shapes, they can define an opening on each of their respective four edges.

For example, the intermediate layer 710 can be formed of a suitable permeable material such as paper, porous plastic, gel, porous oxides, beads and porous ceramic material. The permeable material can permit flow of liquid along at least a length of the intermediate layer 710. For example, liquid can flow through the permeable intermediate layer 710 by capillary movement. For example, in some exemplary embodiments, permeable intermediate layer 710 is also formed of a suitable material that permits exchange of air along at least a length of the intermediate layer 710.

The intermediate layer 710 defines at least one microfluidic chamber 712, which is inserted with the at least one type of microorganism or biological material 9. For example the at least one type of microorganism or biological material 9 can be inserted during the fabrication process of the slide 702. For example, the at least one type of microorganism or biological material 9 can be inserted prior to the intermediate layer 710 being coupled to both the first substrate 704 and second substrate 706.

For example, the at least one microfluidic chamber 712 can be positioned to be aligned with the at least one transparent portion of the first substrate 704 and with the at least one transparent portion of the second substrate 706. Accordingly, for example, light that is transmitted through the first substrate 704 will be received at the at least one microfluidic chamber 712. Light emitted from the microfluidic chamber 712 will pass through the second substrate 706.

For example, the microfluidic chamber 712 can further comprise two electrodes for taking electrical measurements inside the microfluidic chamber. For example, the electrode 721 can be supported against an optical filter 740. A porous membrane (not shown) can optionally be disposed between the electrodes 721 and the optical filter 740. For example, the electrodes can be formed of a plurality of members of a conductive nanomaterial. The nanomaterials can be interweaved to define a plurality of pores that allow passage of liquid through the electrode. For example slide 702 can further comprises any suitable electrical contact for sending and receiving signals to and from the electrodes. For example, at least one input-output conductive lead can be placed on an outer surface of the slide, such as surface 720 of first substrate 704 or surface 722 of second substrate 706. When fabricating the slide 702, a conductive line can be drawn between the electrodes and the input-output conductive lead. The chamber 712 can further comprises food or nutriments for the at least one type of microorganism or biological material 9. Other additives such as preservatives or gels can also be present in the chamber 712.

For example, as the first substrate 704 and the second substrate 706 define at least a first opening 714, at least a region 730 of the intermediate layer is left exposed. When a liquid contacts the exposed region 730, the liquid will permeate through the intermediate layer 710, for example by capillary movement, to reach the microfluidic chamber 712. For example, a water sample can be deposited to contact the exposed region 730. The water sample then permeates through the intermediate layer 710 to reach the microfluidic chamber 712 and mixes with the microorganism or biological material held therein to form a composition. Measurements of at least one electrical property and/or light emitted from the microfluidic chamber will provide indications of the pollution level of the water sample. The at least one electrical property can be measured by means of electrodes 721 that are disposed one beside the other. For example, apparatus 700 can comprise the slide 702 and the at least one light source 30 for emitting light into the at least one microfluidic chamber 712. For example, the at least one light source 30 can be coupled to and supported by the second substrate 706. The apparatus 700 can further comprise at least one photodetector for detecting light emitted from the at least one microfluidic chamber 712. For example, the at least one photodetector 52 can be coupled to and supported by first substrate 704.

Referring now to FIG. 8, therein illustrated is an exemplary apparatus 701 having three microfluidic chambers 712, each having a composition comprising members of the at least one type of microorganism or biological material 9 and a water sample to be evaluated. Each of the three light sources 30 is aligned with one of the microfluidic chambers 712 and one of the three photodetectors 52.

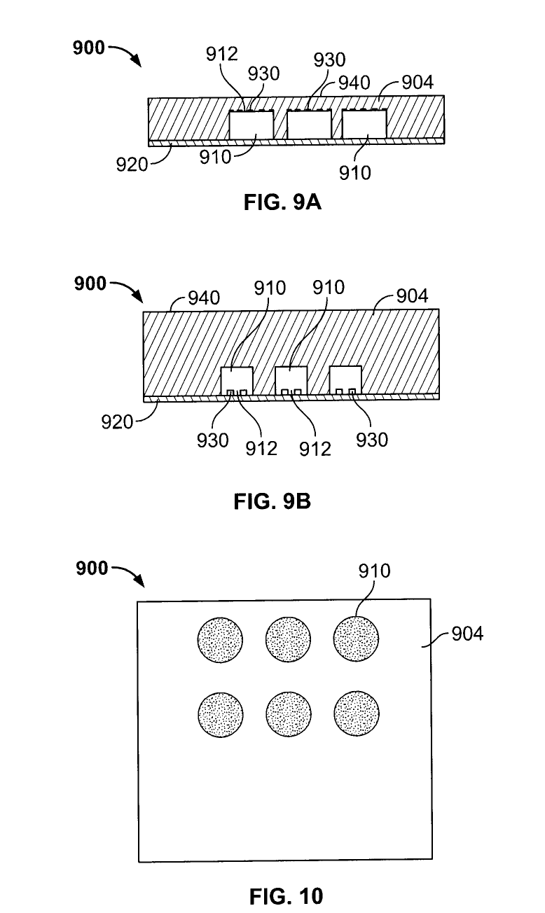

Referring now to FIGS. 9A and 9B, therein illustrated is a side section view of a slide 900 for evaluating a level of pollution according to various exemplary embodiments. Slide 900 comprises a rigid substrate 904 that defines at least one microfluidic recess 910. The at least one recess 910 can hold at least one type of microorganism or biological material 9. The rigid substrate 904 is also semi-transparent or substantially transparent at least at the location of the microfluidic recess 910. For example, the rigid substrate can be formed of glass, transparent polymer, transparent ceramic material or transparent oxide.

At least one opening 912 of microfluidic recess 910 can be covered by a suitable porous material 920 that permits flow of water into the recess while substantially preventing members of the at least one type of microorganism or biological material 9 held in the recess from escaping. The porous material 920 can be a membrane effective for preventing solid particles of a predetermined size from entering into the at least one opening 912. For example, the porous material 920 can be a filter having dimensions similar to the filter 20 and being formed of the same material as filter. For example, the porous material can be a transparent and permeable paper.

The microfluidic recess 910 comprises at least two electrodes 930 for taking at least one electrical measurement. For example, the electrodes can be supported by a side wall or bottom wall of the microfluidic recess 910. For example as shown in FIG. 9A, at least one of the electrode 930 is fixed to the bottom wall of the microfluidic recess 910. Alternatively, as shown in FIG. 9B at least one of the electrode 930 is fixed to the porous material 920. For example, the at least one electrode 930 can comprise a conductive nanomaterial. The nanomaterial can be arranged in a plurality of members defining a plurality of pores for allowing passage of light and water therethrough.

For example slide 900 can further comprises any suitable electrical contact for sending and receiving signals to and from the electrodes. For example, at least one input-output conductive lead can be placed on an outer surface of the slide, such as surface 940 of rigid substrate 904. When fabricating the slide 900, a conductive line can be drawn between the electrodes and the input-output conductive lead.

The substrate 904 can be porous or not. An additional layer can be provided on top of surface 940 (nor shown). This extra layer can be a porous membrane. It can also optionally be a rigid substrate.

Referring now to FIG. 10, therein illustrated is a top view of the slide 900 according to some exemplary embodiments. For example, a plurality of microfluidic recess 910, each having a circular cross section are arranged in a side by side manner in the substrate 904. For example, microfluidic recesses can be manufactured by boring the substrate 904 for a portion of the thickness of the substrate 904. The recesses 910 comprising the at least one type of microorganism or biological material 9.

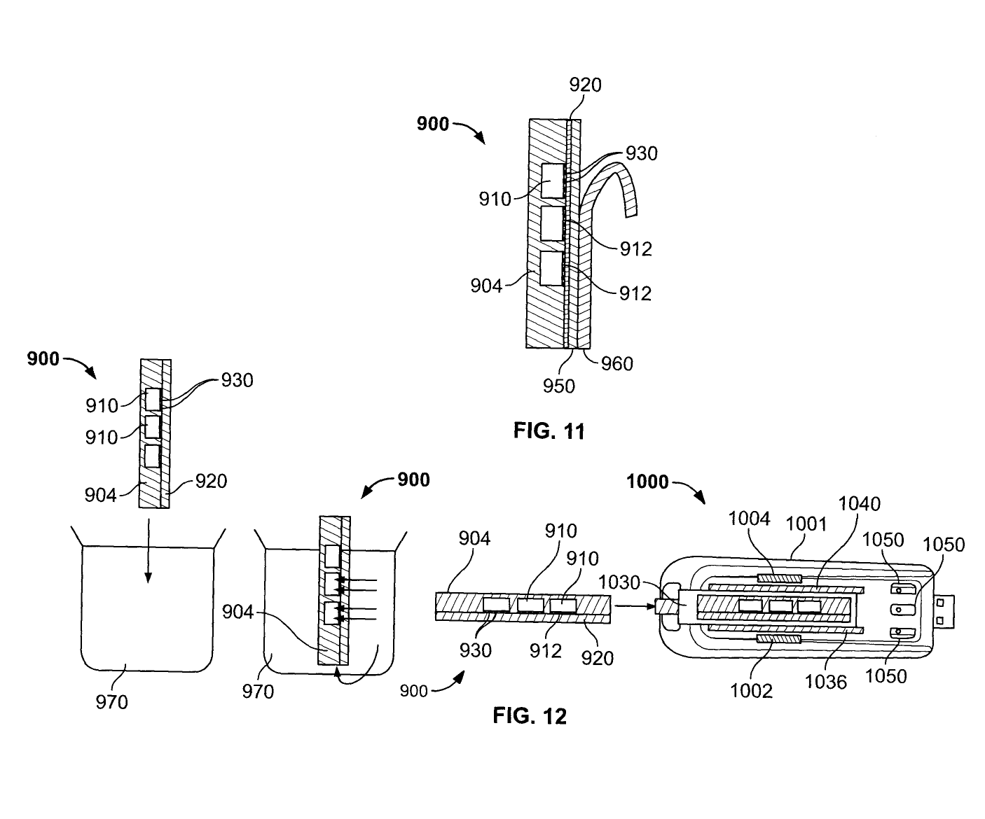

According to some exemplary embodiments, as shown in FIG. 11, the slide 900 can further comprise a first detachable membrane 950 that is connected to the rigid substrate 904 or the porous material and covers the at least one opening of the at least one microfluidic recess 910. For example, the first detachable membrane 950 is also porous for permitting flow liquids. For example pores of the first detachable membrane 950 can be smaller than the pores of the porous material 920. As a result, the detachable membrane 950 can be more opaque than the semi-transparent or substantially transparent porous material 920. The smaller pores of the first detachable membrane 950 substantially prevent larger particles in a volume of water from entering into the microfluidic recess 910 when the slide 900 is submerged into the water.

Referring to FIG. 11, therein shown is a side view of the slide 900 according to some exemplary embodiments. For example, the slide 900 can further comprise a second detachable membrane 960 that can be coupled to the first detachable membrane 950. For example, the second detachable membrane 960 can be formed of a material that is impermeable, but allows exchange of air therethrough. For example, the second detachable membrane 960 can comprise Teflon, hydrophobic polymer like hydrophobic PS, PE, PVDF, PTFE. It can also be any types of treated polymers that are hydrophobics. The second detachable membrane 960 substantially prevents any liquids from entering the microfluidic recess 910 and mixing with the microorganism or biological material s in the recess 910. This is useful when the slide 900 is to be stored and is not being used for evaluating water pollution levels. The exchange of gas or air provided by the second detachable membrane allows microorganism or biological material s, for example microorganisms, held within the microfluidic recess 910 to access CO₂ and other gases that can be vital to the survival of the microorganisms. Again, this aids in the storage of the slide 900 when it is not being used. The membrane material 950 can be a membrane effective for preventing solid particles of a predetermined size from entering into the at least one recess 910. The membrane 960 covering the membrane 950 can thus be permeable to gases but being impermeable to liquids.

Referring now to FIG. 12, therein illustrated is an apparatus 1000 for evaluating water pollution according to exemplary embodiments. Apparatus 1000 comprises a housing 1001 connected to at least one light source for emitting light 1002. For example, the at least one light source can emit light that excites at least one type of photosynthetic microorganism. The apparatus 1000 further comprises at least a photodetector detecting light 1004 connected to the housing 1001. For example, the photodetector 1004 can be configured to detect light in a spectral range corresponding to the range of fluorescent light emitted by excited the least of type of photosynthetic microorganisms. The at least one photodetector 1004 and the at least one light source 1002 defining a space therebetween that is adapted to receive a slide containing a composition to be evaluated and comprising a water sample at the least one type of microorganism or biological material. The apparatus 1000 can be provided with at least one first optical filter 1036 and at least one second optical filter 1040.

Where both the at least one photodetector 1004 and the at least one light source 1002 are planar and are positioned to be substantially parallel, they can be spaced apart in a direction transverse their planes.

The space 1030 defined between 1002 and 1004 is suitably sized to receive a slide used for evaluating pollution level in the water sample. For example, the slide can be any one of the slide described herein, such as chip 4, slide 702 or slide 900.

For example, suitable alignment mechanisms and/or retaining mechanisms can be provided in the apparatus 1000 such that when a slide is received in the space 1030, the at least one microfluidic chamber 8, 812 or 910 of either chip 4, slide 900 or slide 702 can be positioned to be in alignment with the at least one photodetector 1004.

Furthermore, according to some exemplary embodiments, at least one input-output lead can be located on an outer surface of the housing 1001. The positioning of the input-output lead corresponds to the location of the input-output lead on the slide such that when the slide is received in the space 1030 and is positionally aligned, the input-out lead of the slide contacts the input-output lead of the apparatus 1000. Data, control, and/or power signals can then be exchanged through the contacted input-output leads. For example, control signals can be sent from the apparatus 1000 to control the measurement of the at least one electrical property using the at least one electrode of the slide. For example, measured electrical properties can then be sent from the slide as data signals to be received at the apparatus 1000. The apparatus 1000 can also be provided with at least one electrode for measurement of the at least one electrical property.

According to some embodiments, the apparatus 1000 can further comprise a controller for controlling the taking of measurements. For example, the controller is similar to the controller described herein with reference to apparatus 2 and FIGS. 1-6. The controller can be configured to control the at least one light source 1002, the at least one photodetector 1004, and send control signals to and receive data signals from the slide received in the space 1030. For example, operation of the apparatus for taking various measurements can be controlled by a user with at least one external buttons 1050.

For example, the apparatus 1000 can further comprise input-output port that is connected to either the controller, or directly to the at least one light source and the photodetector. For example, the input-output port can be a USB port, but can be any port suitable for connecting to an external device. For example, the input-output port can be used to download data regarding the measured electrical properties and detect light levels to the external device, such as a personal computer.

According to some embodiments, the controller can be a control module being executed on the external device to which the input-output port of the apparatus 1000 is connected. In such cases, apparatus 1000 can receive control signals from the control module via the input-output port, which then further controls the taking of various measurements using the apparatus 1000.

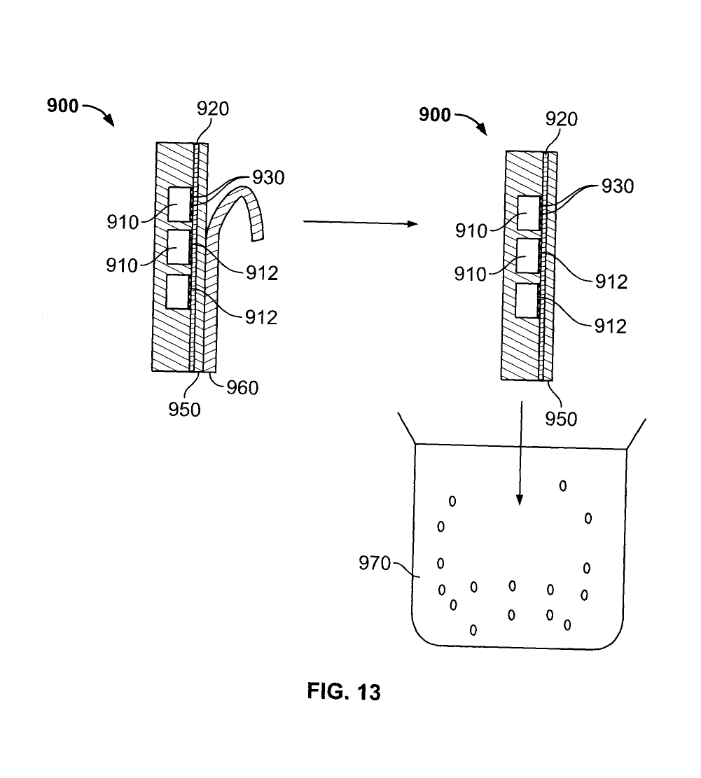

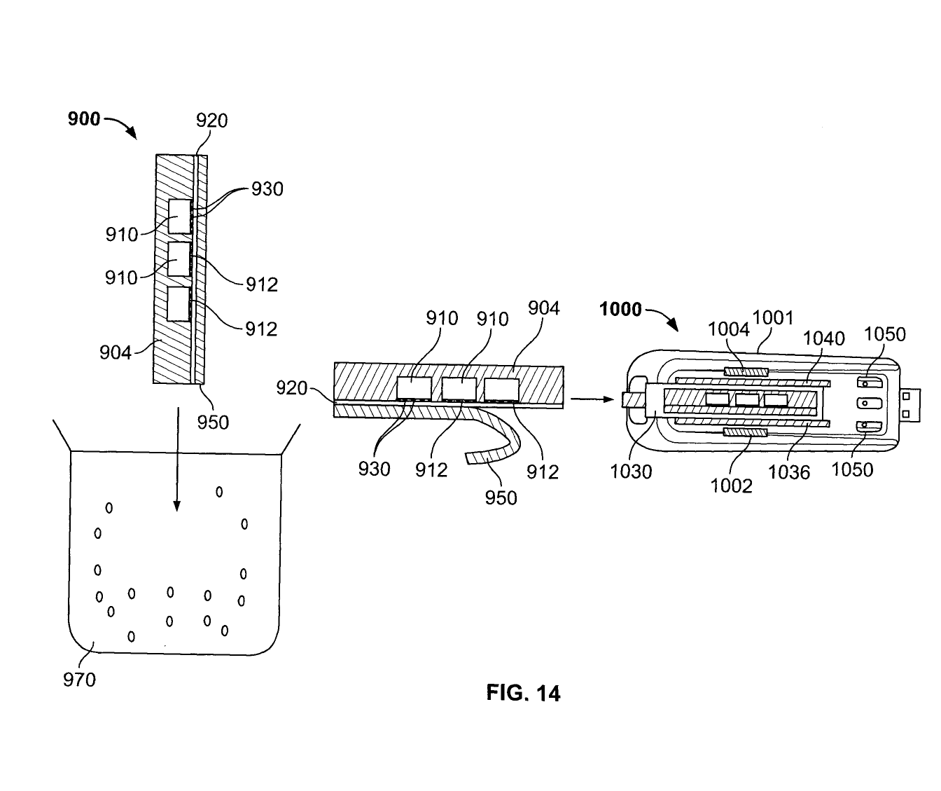

Referring now to FIGS. 13 and 14 together, therein illustrated is an exemplary embodiment of steps of a method for using the slide 900 in conjunction with apparatus 1000 for evaluating a pollution level of a water sample. For example, the slide 900 comprises the at least one type of microorganism or biological material 9 can be stored with both the first detachable membrane 950 and the second detachable membrane 960 still attached to the substrate 904.

Prior to evaluating the level of pollution of water 970, the impermeable second detachable membrane 960 (permeable to gases but impermeable to liquid) is detached from the slide 900. As a result, microfluidic recess 910 is now in liquid communication with the surrounding atmosphere through the porous membrane 920 and the porous first detachable membrane 950 (permeable to both liquid and gases). After having detached the second detachable membrane 960, the slide 900 is submerged into the water 970 to be evaluated. A water sample flows through the porous first detachable membrane 950, the porous membrane 920 and into the microfluidic recess 910 to form a composition with the at least one type of microorganism or biological material 9 held within the microfluidic recess 910.

Continuing with FIG. 14, the slide 900 is then removed from the volume of water 970. The first detachable membrane 950 is then detached from the slide 900. As a result, the side of the slide 900 where the at least one opening 912 of the microfluidic recess 910 is semi-transparent or substantially transparent since the porous material 920 is semi-transparent or substantially transparent. The slide 900 is then inserted into the space 1030 of apparatus 1000 and positioned such that the microfluidic recess 910 is in alignment with the at least one light source 1002 and the photodetector 1004. At least one measurement of the composition can then be taken according to any of the suitable methods described herein.

It will be appreciated that as apparatus 1000 can be adapted to be used with either chip 4, slide 702 or slide 900, it is possible to form a kit comprising the apparatus 1000 and at least one of the chip 4, slide 702 and slide 900.

### Experimental Test

According to one exemplary embodiment of the apparatus and method described herein, a custom-built test apparatus was provided to test the design of the apparatus and system.

According to the test apparatus, a PDMS microfluidic chip was placed on top of a 1 mm thick glass slide. A blue organic light emitting diode made from 4,4′-Bis-(2,2-diphenyl-ethen-1-yl)-biphenyl (DPVBi) was directly placed underneath the detection chamber to excite algal preparations. Algal compositions were exposed to a pollutant solution and then introduced in the microfluidic chamber. A filter (excitation filter) was placed between the OLED and the microfluidic chamber in order to cut the part of the OLED emission that could affect the fluorescence measurement. A second filter (emission filter) was placed between the microfluidic chamber and the photodetector in order to remove the remaining light emitted from the OLED and which was not absorbed by the algae in order to only detect the fluorescence signal from the chlorophyll. A PTB3/1-(3-methoxycarbonyl)-propyl-1-phenyl-(6,6)-C61 (PCBM) blend photodetector was placed on top of the microfluidic chamber to sense the fluorescent light.

According to the test apparatus, the microfluidic PDMS chip was fabricated using standard soft lithography techniques. A SU8-2150 photoresist was used to achieve a 1 mm-deep microfluidic channel. To silanize the mold and allow the peeling of the PDMS from it, few drops of tridecafluoro-1,1,2,2-tetrahydrooctyl-1-trichlorosilane (UCT Inc.) were evaporated on a hot-plate in a closed petri dish for 6 hours at 80° C. Pre-polymer of PDMS was mixed with a cross-linking agent (kit Silgard 184, Dow Corning) at a 10:1 ratio. The devices were fabricated by bonding two parts. The top part was made from the cured PDMS cast on the photoresist molds then pulled off, and the second part was a cover slip made with cured PDMS spin-coated at 4000 rpm. Several microfluidic chambers (up to 16) of 1 mm-deep and 4×3 mm size were fabricated in a single glass substrate (1 mm thick). 24 OLED and OPD junctions of 3×3 mm were fabricated in each single illumination and photodetection devices. Microfluidic chip and OLED based illumination device patterns were designed in order that each pixel aligns directly at the center of the detection chamber once both components assembled.

According to the test apparatus, the blue OLEDs were fabricated on indium tin oxide (ITO) coated glass substrates by multilayer thermal evaporation. Organic small molecules materials: 2,9-dimethyl-4,7-diphenyl-1,10-phenanthroline (BCP), N,N′-bis(naphthalen-1-yl)-N,N′-bis(phenyl)-benzidine (NPB), Tris(8-hydroxy-quinolinato)aluminium (Alq3) and DPVBi purchased from Lumtec™ were used without further purification. The ITO coated substrates were patterned and cleaned using conventional procedures with solvent and oxygen plasma. Successive layers of NPB (hole injection layer, 50 nm), DPVBi (emitting layer, 30 nm), BCP (hole blocking layer, 5 nm), Alq3 (electron injection layer, 35 nm), LiF (1 nm) and Al (100 nm) were then deposited using a vacuum evaporator. The PTB3 conductive polymer was used for the fabrication of the organic photodetector. This polymer was synthesized. To fabricate the OPD, the active layer was made of a 1:1 blend of PTB3 and PCBM in chlorobenzene (with 3% in volume of 1,8-diiodooctane). The blend was deposited on top of an ITO coated glass substrate by spin coating. Finally, the cathode was formed by depositing 1 nm of LiF and 100 nm of aluminum using thermal vacuum evaporation. The organic devices were encapsulated by placing a glass cover fixed by UV cured epoxy on top of the active area. The encapsulation was done in a nitrogen glove box right out after removing devices from the thermal evaporator to prevent air and humidity device degradation. OLED emission spectrum was collected with an USB2000 (Ocean Optics) spectrometer. External quantum efficiency (EQE) was measured with a Keithley 2601a™ source measure unit. For those measurements, the device was illuminated by the light from a xenon lamp passing through a monochromator (Cornerstone 130 ⅛ M, Oriel) with an intensity of about 20 μW. A calibrated silicon diode with known spectral response was used as a reference. According to the test apparatus, the emission and excitation filters were fabricated by incorporating dyes in a host resin. The emission filter is composed of a set of acid/basic dyes. Acid yellow 34, acid red 73 and basic violet 3 at 20, 20, 10 mg/mL respectively, were mixed separately in a fish gelatin resin. Each individual mixture was then successively spin coated, one on top of the other, on 100 μm thick glass substrates. To fabricate the excitation filter, (TOMA)2CoBr4 compound has been synthesized. The viscous preparation was taken in sandwich between two 100 μm thick glass substrates and sealed with epoxy to protect it from humidity.

According to an experimental evaluation using the test apparatus, green algae Chlamydomonas reinhardtii (CC-125) was cultivated in 250 mL Erlenmeyer flasks in High Salt Medium (HSM) with the adjusted pH=6.8±0.1. The algae were grown at 25° C. under a light intensity of 100 μmol·m-2·s-1 provided by white-light neon lamps and a 16 h-light/8 h-dark cycle. Cells were maintained continuously in the mid-exponential growth phase (up to 4×106 cell/ml) before experiments. To measure the minimum density of algae that can be detected, successive dilutions of a 3×106 cell/ml algal culture were prepared in HSM. These solutions were dark adapted for 15 min before fluorescence measurement in order to reoxidize photosystem II reaction centers.

According to the experimental evaluation using the test apparatus, pollutant detection measurements, a 1×106 cell/ml green algal culture was used. Different concentrations of Diuron or DCMU (3(3,4-dichlorophenyl)-1,1-dimethylurea from Aldrich) were prepared in pure ethanol. For each measurement, 30 μL of DCMU was mixed with 2 mL of algal solution. The mixtures were exposed for 30 min under a 100 μmol·m-2·s-1 light intensity, and then dark adapted for 15 min before being injected into the microfluidic chip with a syringe pump to fully fill a microfluidic chamber (around 10 μL). Algal exposure to ethanol concentration used in this study (without DCMU) had no effect on the fluorescence measurements (data not shown). Each measurement was replicated three times. The OPD was operated in the photovoltaic mode under zero bias, the pulsed OLED was used for the excitation. Photocurrent was converted by a current/voltage amplifier (Analog Devices AD549) and fed into the voltage port of an acquisition card (USB-1408FS) at 1 kHz. Between each measurement the microfluidic chamber was cleaned by flushing with ethanol and water for several times.

According to the experimental evaluation using the test apparatus, Handy-PEA fluorometer (Hansatech Ltd.) was used as the commercial available equipment to be compared with the microfluidic sensor. To do so, the same 1×10⁶ cell/ml green algal culture (cultivated under the same environment) has been treated under the same experimental conditions like before. The Handy-PEA system uses three ultra-bright red LED's providing excitation light with a maximum emission at 650 nm (spectral line half width of 22 nm). Fluorescence emission was detected for wavelengths over 700 nm.

The test apparatus, had a thickness that essentially depends on the thickness of the used substrate. In fact, each organic device has been fabricated on a 1.1 mm thick ITO coated glass slide and the microfluidic chip on a 1 mm thick glass slide in order to get mechanical strength during the fabrication process. Thus the total thickness is about 4 mm.

According to the test apparatus, the surface dimension of the chip was about 5 cm square, which only depends on the total amount of chambers that includes the chip. In the test apparatus, the organic optoelectronic devices included more than 24 active elements to be used with microfluidic chips of 8-16 chambers each. With these characteristics, 24 series of measurements with the same organic devices was possible. Thus, organic devices, combined with microfluidic chip technology, are a suitable solution to integrate several microfluidic chambers into the chip.

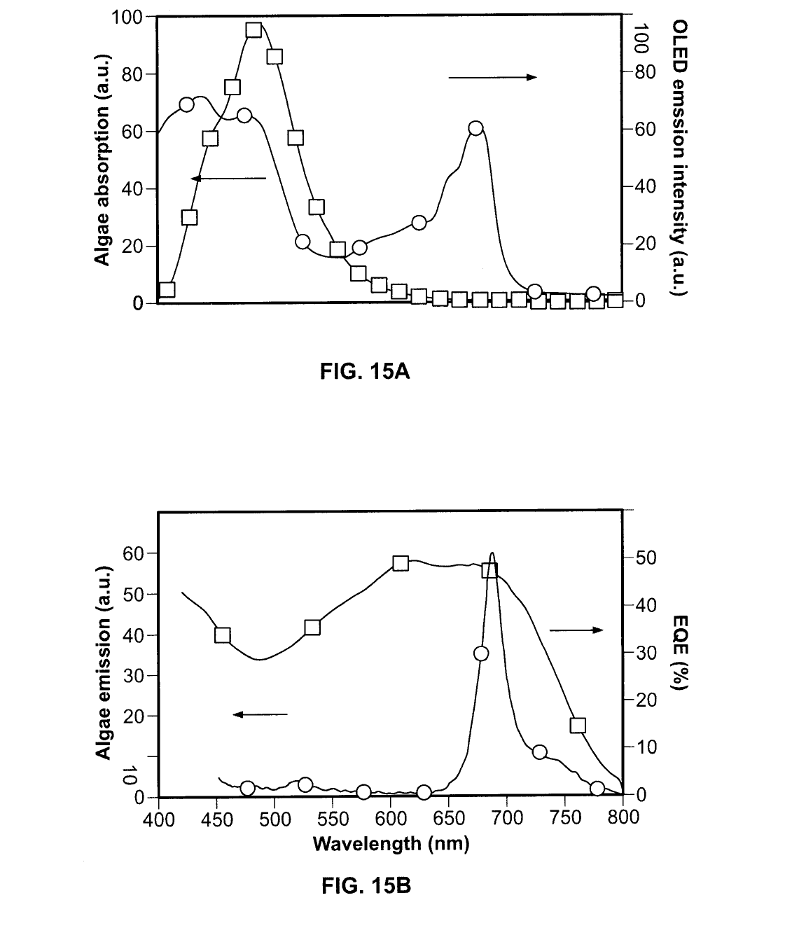

According to the experimental evaluation using the test apparatus, as shown in FIG. 15A, green algae have two absorption spectral ranges situated at 400-500 nm and 650-680 nm. This absorption is essentially due to the chlorophylls and the carotenoids. As shown in FIG. 15b, green algae only have fluorescence emission with a peak situated around 685 nm. All the excess energy absorbed by algal pigments that is not used for photosynthesis is reemitted as heat or is transmitted to chlorophyll a and reemitted as fluorescence originating from chlorophyll a (between 680-720 nm).

FIG. 15A is an absorption spectrum of the green algae CC125 and the blue OLED emission spectrum. FIG. 15b Fluorescence emission spectrum of the green algae CC125 and the external quantum efficiency of the PTB3/PC61BM OPD at 0V.

According to the experimental evaluation using the test apparatus, and as shown in FIG. 15A, there are two possibilities to excite green algae using blue (400-500 nm) or red (650-680 nm) light within the test apparatus. When using an OLED for illumination the use of a blue light offered two major advantages. First, considering that OLED have large bandwidth (about 100 nm) emission spectra, a blue OLED was more efficient to excite green algae. In fact, for a red OLED, the optical filter (excitation filter) needed to avoid overlap with the fluorescence emission will cut half of the absorption peak in the red region. Second, because there is a large gap between the blue and red region, sharp cutoff optical filters are not necessary in this case and the use of absorption filter could be a suitable solution. Thus, a blue OLED made from DPVBi was used. Its emission spectrum is shown FIG. 15A. As can be seen in this figure, it has an emission peak situated at around 485 nm, which nicely overlaps one of the spectral absorption range of the algae. The fabricated blue OLED had a high performance in terms of luminescence as more than 10,000 Cd/m² could be reached. However, pulse tests with different pulse times (0.5 s to 20 s) and intensities showed that OLED performance greatly decreases when used at maximum operation voltage and current density. Therefore, the operation pulse voltage was fixed at 12 V, corresponding to a light intensity of 4,700 Cd/m². In these conditions, no noticeable decrease of luminescence was observed during the course of the experiments.

FIG. 15b shows the external quantum efficiency (EQE) of the OPD according to the test apparatus. It will be appreciated from this figure, the near-infrared solution process OPD had a broadband photo response from 600 to 700 nm and entirely covered the algal fluorescence emission. Its sensitivity at 685 nm, which is the maximum peak of the algal fluorescence emission, was 0.26 A/W (corresponding of an EQE of 47%) while its dark current density at 0 V was lower than 1 nA/cm2. Its time response of 1 μs is sufficient for algal fluorescence. These characteristics place it among the most sensitive OPD between 600 nm and 700.

According to the test apparatus, the OLEDs were aligned with the OPD in order to get the maximum fluorescence signal. However, in this configuration, due to the large spectral range of the OLED emission, as shown in FIG. 15A, overlapping of this emission with the fluorescence emission from the algae could occur. Moreover, as the emission from the OLED is not completely absorbed by the algae, some residual light from the OLED could reach the OPD. In order to avoid these problems, it two optical filters were used as in some embodiments shown in FIG. 1.

According to the test apparatus, the filters to be integrated should exhibit limited auto-fluorescence, high transmittance at the desired wavelength, high attenuation of unwanted wavelengths, and should be inexpensive to fabricate. Available technologies include interference filters, absorption filters and polarizing filters. For this application, interference filter fabrication is too expensive. A microfluidic sensor is not ideal for the current application: polarizing filters absorb more than 60% of light, while dye doped PDMS could have a toxic effect on algae. For these reasons, it was chosen to integrate a dye-doped resin that could easily be fabricated by spin coating.

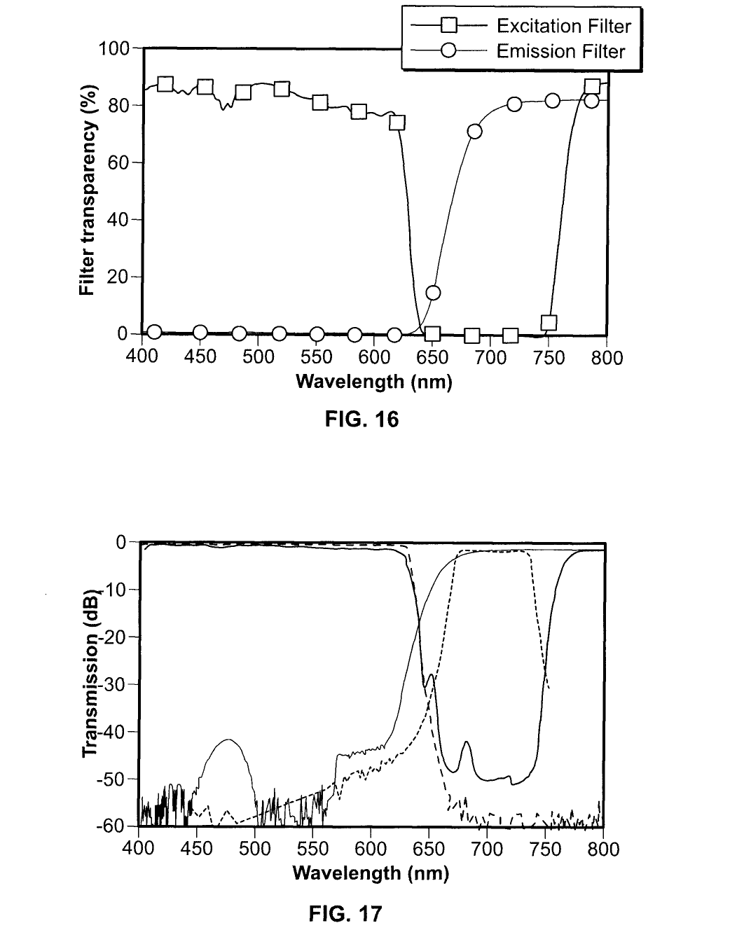

FIG. 16 is a transmittance spectra of the fabricated excitation (blue line) and the emission (red line) filters.

According to the test apparatus, acid/base dyes were used for the fabrication of the emission filter because of their large commercial selection and low cost. Moreover, these dyes offer the advantage that their absorption ranges can be modulated by incorporating different dyes. Optimization of the dyes compositions and concentrations lead to a final filter made from three components, yellow 34, acid red 73 and basic violet 3 with three appropriate concentrations. FIG. 16 shows the optical spectral transmission of this filter. It shows that achieved a long-pass filter with a cut-off wavelength of 667 nm and with a transmittance of more than 75% at the peak of algae fluorescence emission (685 nm) was achieved.

According to the test apparatus, for the excitation filter, placed between the OLEDs and the microfluidic chambers, a different approach had to be taken as the desired absorbance range of 650-750 nm could not be achieved with acid/base dyes without significant absorbance in the 400-500 nm spectral range. To circumvent this, a dye-doped resin was prepared with a metal complex capable of absorbing strongly in the 650-700 nm range, while simultaneously maintaining transmittance in the 400-500 nm wavelengths. After experimenting with various metal complexes, the excitation filter was fabricated by using the Co2+ doped resin coming from the (TOMA)2CoBr4 compound. The fabricated short-pass excitation filter has a cut-off wavelength of 626 nm (FIG. 16) and can then cut the extra emission spectrum from the OLED that could overlap the fluorescence emission from the algae at 685 nm. Moreover, high transmittance with more than 80% was obtained.

According to the test apparatus, as a result, the completed dye-doped filters have high absorbance in the desired wavelengths, yet high attenuation in the undesired ones. FIG. 17 shows the comparison of the transmission spectra of the filters with commercial interference filters. The dye-doped filters had quite similar characteristics, although the cut-off was not as sharp. Nonetheless, the obtained attenuation was good enough that no more polarizing filtering was needed.

According to the test apparatus, in both cases, emission and excitation filters, the total thickness of filters did not exceed 1-10 μm, not including the 100 μm thick glass substrates, which make them perfectly suitable for their integration on the thin planar configuration of the current photodetector.

FIG. 17 Transmittance spectra of the fabricated excitation (blue line) and the emission filters (red line) compared to the commercial excitation (blue dashed line) and emission (red dashed line) filters.

According to the test apparatus, silver nanofilaments are synthesized in ethylene glycol at 160 degrees from polyvinyl pyrrolydone, silver nitrate and copper sulphate. Further to cleaning steps, filaments (10-100 um long and ≈100 nm wide) are dispersed into alcohol, forming a stable liquid ink. A small amount of nanofilaments is filtered on a filtering membrane, forming a conductive porous electrode on the filtering medium. The electrode is then transferred by stamping on a chemically treated glass sheet to improve electrode adherence. The electrode formed as result of this process can be working electrode 61, counter electrode 62, reference electrode 63, or a combination thereof.

From this transparent porous macro electrode on the glass sheet, the electrodes are built by lithography. Lithography steps include a step of protection by a protective photosensitive resin, which is then followed by engraving and deprotecting steps. Semi-transparent electrodes made of silver nanofilaments are formed. Two of the three electrodes can be covered with electro-deposited platinum, copper or gold. For example, platinum can be used. In some cases, non-transparent material, such as gold, can be used for the counter electrode.

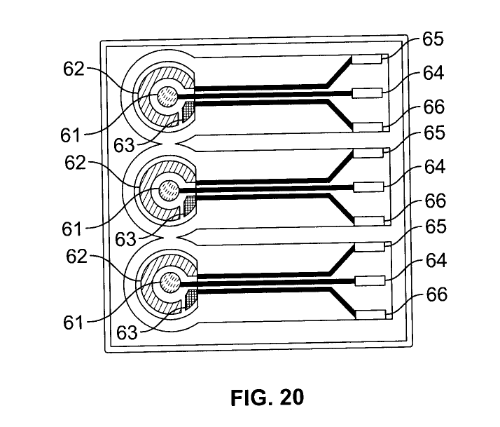

Referring now to FIG. 20, therein is a plan view of three electric detectors each having a working electrode, counter electrode and reference electrode fabricated according to the process described in relation to the test apparatus. It will be appreciated that the working electrodes 61 have a substantially circular shape. The counter electrodes 62 have an elongated shape defining a circular arc.

According to the test apparatus, the working electrode 61 has an area of 4 mm², the counter electrode 62 has an area of 10 mm², and the reference area has of 1.6 mm². Leads and electrical lines connecting the electrodes with the leads can be covered by a polymer resin for protection. Accordingly, only the electrodes 61, 62, and 63 are left exposed.

According to the test apparatus, the electrodes are semi-transparent, with a transparency higher than 60% in the desired wavelengths. In some cases, the sheet resistance of the electrodes is less than 10 ohm/square. This is the case for transparency levels that are less than 75%. It was found that coating silver nanofilaments can diminish transparency, and in some cases decrease the transparency level to 58% while increasing resistivity (from 8 ohm/square to 30 ohm/square).

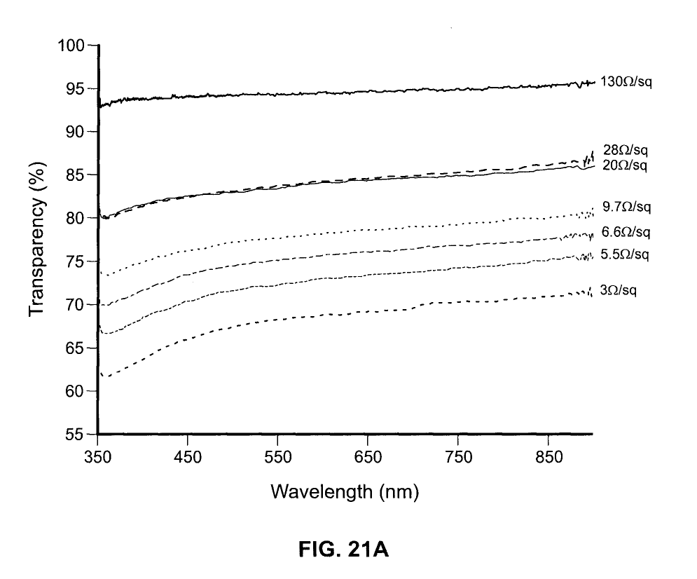

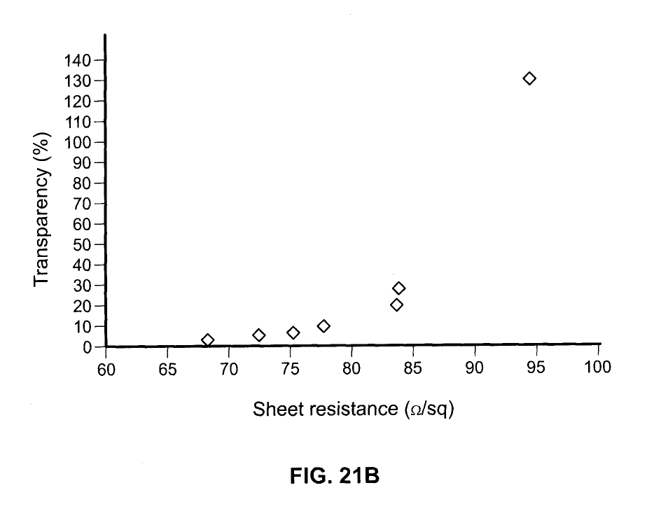

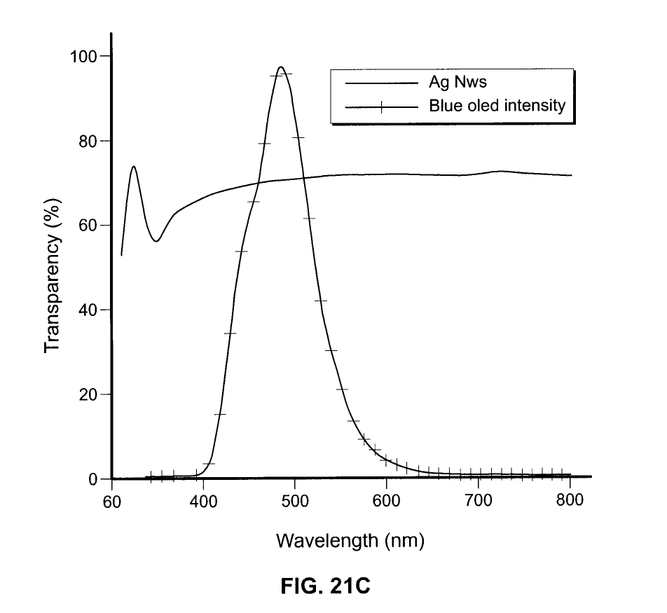

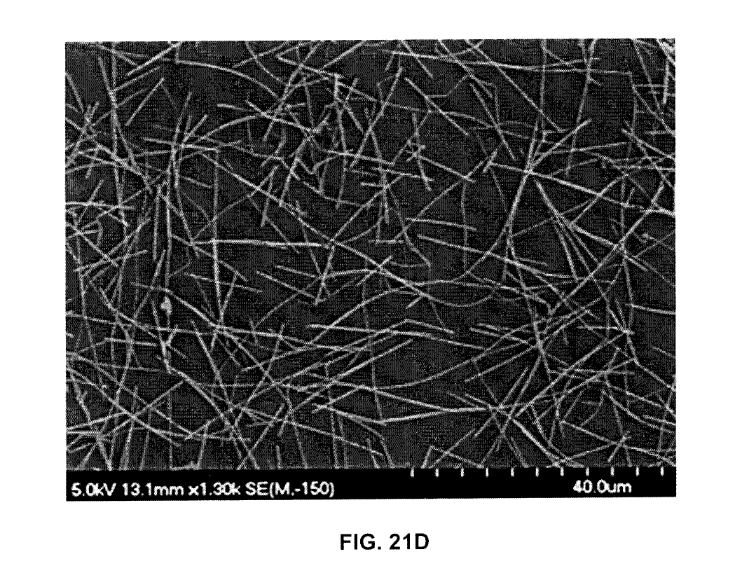

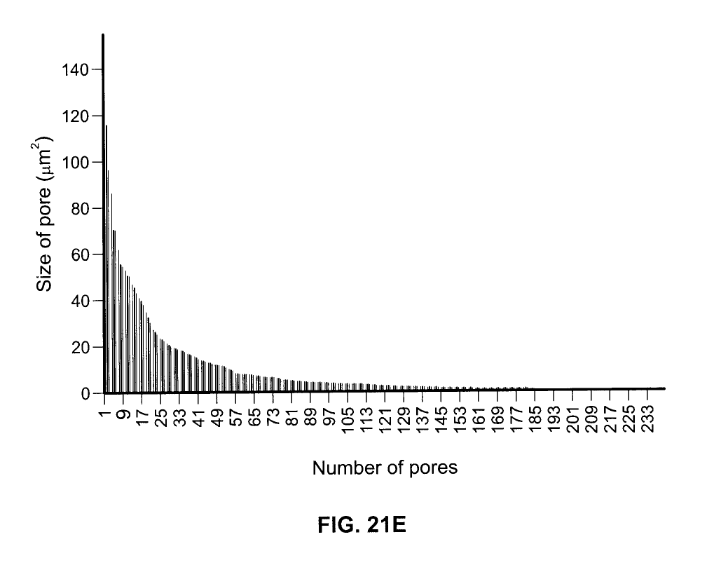

FIG. 21A shows transparency levels of electrodes of different resistivity over the range of desired wavelengths.

FIG. 21B shows sheet resistance of an electrode formed of silver nanofilaments for different transparency levels.

FIG. 21C shows transparency of an electrode formed of silver nanofilaments over the range of desired wavelengths.

FIG. 21D shows a magnification of an electrode taken using a scanning electrode microscope. Pores having a size of 11±10 um² can be achieved.

FIG. 21E shows variations of the size of pores over different number of pores provided in the electrode.

### Algal Fluorescence Measurement

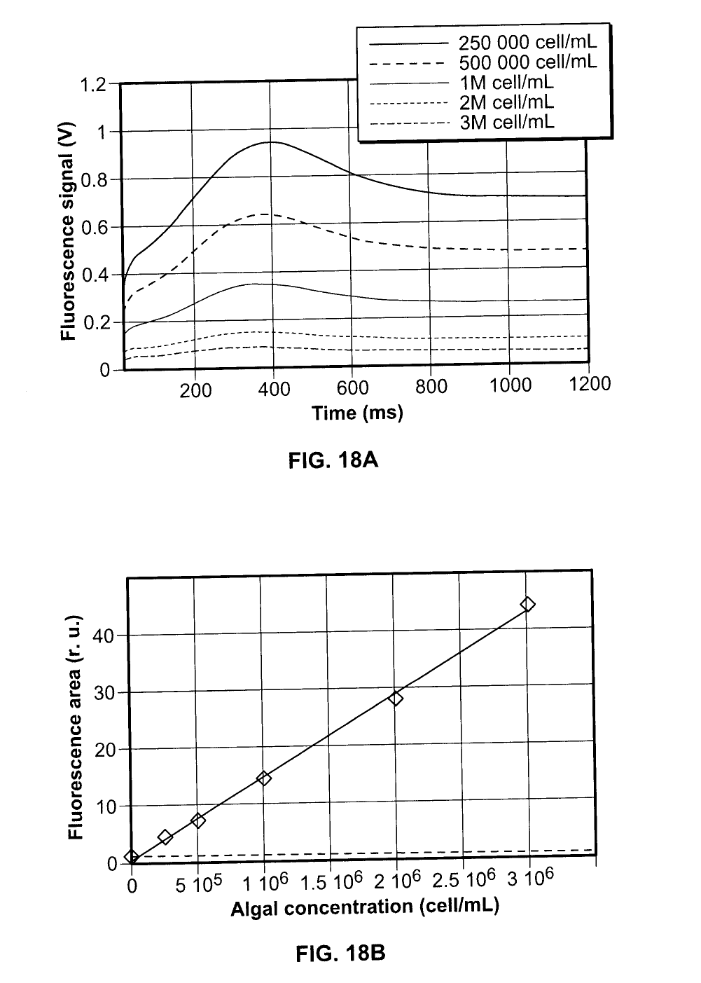

According to the experimental evaluation using the test apparatus, FIG. 18A shows the fluorescence signals detected by the OPD with a 1.2 s OLED pulse at different algal concentrations as a function of time after start of illumination according to the test apparatus and method. Each curve represents algal fluorescence (voltage generated in the OPD by a pulse of illumination in presence of algae subtracted from the dark voltage of the OPD without algae). The first value of fluorescence shown on FIG. 18A for each algal concentration corresponds to the value measured at 25 ms after start of illumination. As can be seen on the figure, for each algal concentration, the fluorescence signal of healthy algae gradually increased to peak at 350 ms and subsequently decreased. The first part of the fluorescence kinetic indicates the progressive closure of PSII reaction centers. After the maximal fluorescence level, fluorescence signal begins to decrease due to photochemical quenching. Indeed, there is an increase in the rate at which electrons are transported away from PSII. It can be observed that the fluorescence intensity increases with algal concentration for all the period of fluorescence emission. Moreover, the blue OLED was able to excite algae with enough photons to induce and detect fluorescence even at relatively low algal concentrations. In fact, fluorescence with as few as 2200 cells in the detection chamber (9 μL detection chamber volume, 250,000 cell/mL concentration) could be measured. From these curves, it is possible to calculate the area under each curve and plotted it in FIG. 18B to visualize the linear evolution of the fluorescence level as a function of the algal concentration. From FIG. 18B it is possible to quantify the algal concentration from a solution when the response of the OPD has been previously calibrated. By taking noise level into consideration (dashed line), a limit of detection of 210,000 cell/mL can be estimated with a ratio S/N=3 for the actual system set-up. This value corresponds to a limit of detection of 1,700 cells in the detection chamber.

According to the experimental evaluation using the test apparatus, FIG. 18A Algal fluorescence response measured with the OPD at different algal concentrations. FIG. 18B Fluorescence area as function of algal concentration (solid line represents the linear fitting curve; dashed line represents the noise limit)

### Herbicide Fluorescence Measurement

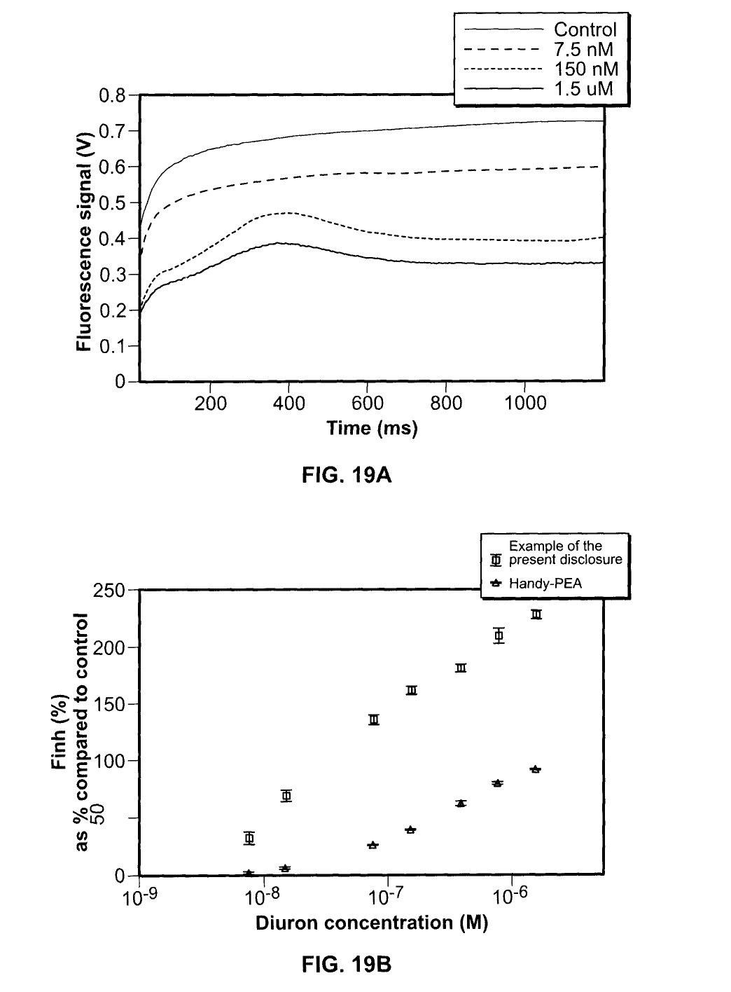

According to the experimental evaluation using the test apparatus, FIG. 19A shows the fluorescence response as a function of time (from 25 to 1200 ms) for algal culture of 1×10⁶ cell/ml concentration exposed to different DCMU concentrations. It was noticed that the injection of the pollutant changes fluorescence kinetics. An increase in the fluorescence signal for the first 100 ms, proportional to the pollutant concentration was observed. DCMU induced this fluorescence increase because it blocks the electron transfer in PSII. The electrons are returning to the PSII reaction centers and the energy is then transfer back to the Chlorophyll to emit fluorescence. As the concentration of DCMU increases, the number of PSII reaction centers closed is higher, resulting in the increase of the fluorescence emitted by the organisms.

FIG. 19A refers to algal fluorescence signal detected with the OPD for different concentration of Diuron. FIG. 19B relates to variation of the inhibition factor (calculated with Vj and F25m) as function of Diuron concentration.

According to the experimental evaluation using the test apparatus, from this kinetic of the fluorescence signal, it is possible to extract several parameters representing physiological processes. Here, two very sensitive parameters (even if different), one for the test apparatus and another for the commercial PEA was extracted. For the commercial equipment, the parameter Vj=(F2 ms−F50 μs)/(FM−F50 μs) were calculated, where F50 μs, FM, and F2 ms are respectively the initial fluorescence at 50 μs, the maximum fluorescence, and the fluorescence measured at 2 ms. The relative variable fluorescence at 2 ms Vj is very sensitive to Diuron as it is proportional to reaction centers closed at 2 ms. For the test apparatus, it was calculated a more suitable parameter F25m=F25 ms/Fmax where F25 ms is the fluorescence at 25 ms and Fmax is the maximal fluorescence value at 1.5 μM of Diuron (maximum herbicide concentration used). In order to compare the sensitivity of the test apparatus with the sensitivity of the commercial system, an inhibiton factor (Finh) based on the fluorescence measurements was calculated. Finh=[parameter C−parameter T]/parameter C, where C and T represent parameter values from control and treated samples, respectively. The two inhibition factors (in percentage), as calculated with Vj and with F25m, have been plotted in FIG. 19B. A higher percentage will indicate a stronger inhibition of photochemistry by the used herbicide, while a lower value will indicate a lower effect of the tested pollutant on photosynthetic efficiency. As the concentration of DCMU increases, it is possible to measure the increase in photosynthesis inhibition following a dose-response curve. 1.5 μM of DCMU was the highest concentration used for maximum photochemistry inhibition with 30 min of light exposure. The lowest concentration of DCMU tested that gave a significant difference with the control algae for the Finh parameter was 7.5 nM. FIG. 19B shows that the inhibitory fluorescence factor of the integrated device is more sensitive than using the commercial equipment Handy-PEA. In fact, the concentration of DCMU inhibiting 50% of the algae photochemistry (EC50) was of 1.1×10−⁸ M for our device (evaluated with F25m) as compared to a EC50=2.2×10−⁷ M for the Handy-PEA commercial system (evaluated with Vj). This result indicates the test apparatus has a high sensitivity for herbicide detection through fluorescence variation. In comparison, using portable electrical biosensors based on algae for the detection of diuron, obtained values of 50% the inhibition ratio of oxygen reduction current IC50=1×10 −⁶ M. This value is 100 times higher than the EC50 established by using the test apparatus. Thus, fully integrated test apparatus based on detecting fluorescence from algae exhibits outstanding sensitivity compared with portable electrical biosensors and transportable commercial fluorescence equipment like the Handy-PEA™.

From these results it is possible conclude than when only herbicide Diuron is present in water, the test apparatus will be able to detect its presence even at low concentrations.

### Oxygen Concentration Measurement

According to an experimental evaluation, in order to measure the oxygen level, the electrodes making up the electrical detector are integrated in a glass microfluidic channel, and aligned on an OLED. Algae culture CC125 (5M cell/ml concentration) is injected in the microfluidic channel, the oxygen measure being continuously taken through applying-0.6V between the working electrode and the reference electrode. A Diuron concentration of 1 uM is added to the algae culture before the injection into the chip and the measuring. Standard measures of 1 uM of pollutant were made in triplicate.

It was found that measurement of oxygen concentration, like measurement of fluorescence, is a parameter that will vary in the presence of pollutant. Oxygen variation of algae, which is the combination of both production and breathing of algae, can therefore be linked to the pollutant concentration contained in the analyte. In order to measure oxygen production, this detector is also composed of the same organic light source used by the fluorescence detector (OLED.

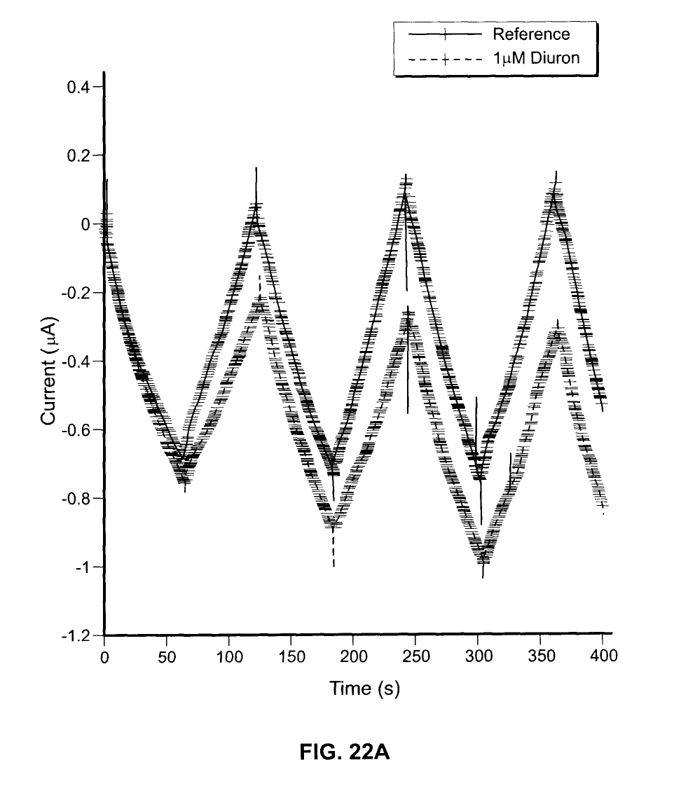

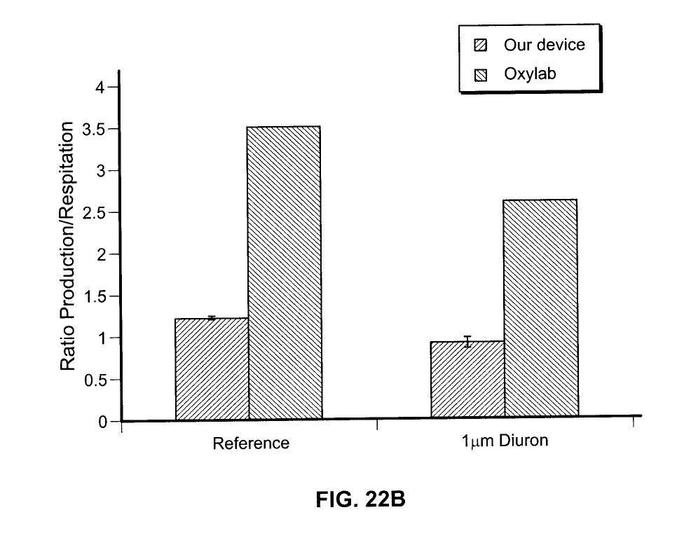

FIG. 22A refers to oxygen concentration levels measured with the electrical detector for 1 μM of Diuron and with a reference (that has not been exposed to Diuron).

FIG. 22B refers to oxygen concentration measured using the test apparatus in comparison with a commercial device (Oxylab).

It was found that addition of 1 μM of Diuron caused an about 26% decrease in total oxygen production from algae.

The examples of methods and apparatuses previously described represent a very significant improvement of the technology for the evaluation of a level of pollution of a water sample by proposing

1. An apparatus comprising components having a small size for quickly evaluating level of pollution of a water sample, thus allowing the apparatus to be portable and, in some cases, disposable and be easily deployable in the field.

2. A method for evaluating level of pollution by detecting emissions of fluorescent light from microorganisms undergoing photosynthesis

The examples of methods and apparatus herein described also offer the following advantages:
1. Integration of several chambers with different algal species on the same microfluidic platform. This integration could be done in order to measure in a single test toxicity of water and detect the presence of several pollutants simultaneously.
2. small intensity fluorescence variation induced by an herbicide pollutant at low concentrations are measurable
3. Miniature size of the components.
4. Lower costs for evaluation of water pollution.
5. Ease of use.
6. Significant lowering of time required to obtain results of an evaluation of the level of pollution water samples.
7. Measurement of light emitted from a composition and measurement of electrical properties of the composition within the same microfluidic chamber.

The scope of the claims should not be limited by specific embodiments and examples provided in the disclosure, but should be given the broadest interpretation consistent with the disclosure as a whole.

1-107. (canceled)

108. An apparatus for evaluating water pollution comprising:
at least one light source for emitting light having a spectral range for causing at least one type of microorganism or biological material to undergo cell activity and emit fluorescent light;
at least one photodetector for detecting a level of fluorescent light;
a chip disposed between the at least one light source and the detector, the chip comprising at least one microfluidic channel disposed for being exposed to light from the at least one light source and dimensioned for receiving a composition comprising the at least one type of microorganism or biological material and a water sample to be evaluated; and
at least one electric detector in the at least one microfluidic channel for detecting at least one property of the composition, said at least one detector comprising at least one electrode;
wherein the detected level of fluorescent light provides a first indication of pollution level in the water sample and the at least one detected property of the composition provides a second indication of the pollution level of the water sample,
and wherein at least one of the electrodes is semi-transparent.

109. The apparatus of claim 008, wherein the at least one microfluidic channel defines at least one microfluidic chamber, the at least one chamber comprising a filter substantially preventing passage of the microorganisms while permitting flow of the water sample therethrough; and wherein the at least one of the electrodes comprised in the electric detector is positioned within the at least one microfluidic chamber.

110. The apparatus of claim 109,
wherein the filter is at least semi-transparent; and
wherein the at least one photodetector, the at least one microfluidic chamber, and the filter are substantially aligned together.

111. The apparatus of claim 110, wherein the at least one light source is aligned with the at least one photodetector.

112. The apparatus of claim 109,
wherein the chip defines a chip plane,
wherein the filter is at least semi-transparent; and
wherein the at least one photodetector, the at least one microfluidic chamber, and the filter are substantially aligned in a direction transverse the chip plane.

113. The apparatus of claim 108, wherein at least one of the electrodes comprises a nanomaterial being connected to the filter, the nanomaterial being arranged in a plurality of members defining a plurality of pores for allowing passage of light and/or water therethrough.

114. The apparatus of claim 108, wherein at least one of the electrodes is porous.

115. The apparatus of claim 108, wherein the at least one of the electrodes has a transparency greater than about 60%.

116. The apparatus of claim 108, wherein the resistance of the at least one electrode is between about 50% and about 70% and wherein the transparency of the at least one electrode is about 8 ohms/square to about 30 ohms/square.

117. The apparatus of claim 108, wherein the at least one microfluidic channel has a depth of less than about 2 mm.

118. The apparatus of claim 117, wherein the chip defines a thickness of less than about 10 mm.

119. A chip for receiving at least one type of microorganism or biological material comprising:
a substrate defining at least one microfluidic channel for receiving a composition comprising a water sample and the at least one type of microorganism or biological material, the at least one microfluidic channel further defining at least one microfluidic chamber, the substrate being substantially transparent at the location of the microfluidic chamber;
a filter that is at least substantially semi-transparent and that is supported within the microfluidic chamber, the filter substantially preventing passage of the at least one of microorganism or biological material while permitting flow of the water sample therethrough, the filter being aligned with a substantially transparent portion of the substrate; and
at least two electrodes positioned within the microfluidic channel for taking at least one electrical measurement.

120. The chip of claim 119, wherein at least one of the electrodes comprises a nanomaterial being connected to the filter, the nanomaterial being arranged in a plurality of members defining a plurality of pores for allowing passage of light and water therethrough.

121. The chip of claim 119, wherein at least one of the electrodes is semi-transparent.

122. The chip of claim 121, wherein at least one of the electrodes is porous.

123. An apparatus for evaluating water pollution comprising the chip of claim 119, the apparatus further comprising:
at least one light source for emitting light; and
at least one photodetector for detecting a light;
wherein the apparatus is adapted to receive the chip between the at least one light source and the at least one photodetector.

124. The apparatus of claim 123,
wherein the at least one type of microorganism or biological material is at least one type of photosynthetic microorganism;
wherein the at least one light source emits light having a spectral range for causing the at least one type of photosynthetic microorganism to undergo photosynthesis and emit excess energy as fluorescent light; and
wherein, the detector is adapted for detecting a level of fluorescent light, the detected level of fluorescent light providing an additional indication of level of pollution of the water sample.

125. The apparatus of claim 123, wherein the at least one photodetector, the at least one microfluidic chamber and the at least one light source are substantially aligned together, the at least one light source being effective for emitting light onto the microfluidic chamber and light emitted from the aligned microfluidic chamber being detected by the photodetector, and wherein the at least two electrodes being effective for detecting the at least one property of the composition in the aligned microfluidic chamber, thereby allowing for measuring simultaneously a first indication of pollution level in the water sample by means of the at least one photodetector and a second indication of the pollution level of the water sample by means of the at least one detected property of the composition detected by the at least one electric detector.

126. An electrode comprising a nanomaterial, the nanomaterial being arranged in a plurality of members defining a plurality of pores for allowing passage of light therethrough, wherein said electrode is substantially transparent.

127. The electrode of claim 126, wherein said electrode allows passage of at least 80% in the about 390 nm to about 800 nm wavelength range.
